(19) United States
(12) Patent Application Publication
(10) Pub. No.: US 2018/0221298 A1
(43) Pub. Date: Aug. 9, 2018

(54) TRANSDERMAL DELIVERY SYSTEM

(71) Applicant: EURO-CELTIQUE S.A., Luxembourg (LU)

(72) Inventors: Steve WHITELOCK, Cambridge (GB); Deborah Phyllis HARDING, Cambridge (GB); Helen JOHNSON, Cambridge (GB); Kevin SMITH, Cambridge (GB); Gill MUNDIN, Cambridge (GB)

(73) Assignee: Euro-Celtique S.A., Luxembourg (LU)

(21) Appl. No.: 15/748,545

(22) PCT Filed: Jul. 28, 2016
(86) PCT No.: PCT/GB2016/052308

§ 371 Date: Jan. 29, 2018

(30) Foreign Application Priority Data
Jul. 30, 2015 (GB) 1513442.2

(51) Int. Cl. A61K 9/70; A61K 9/00; A61K 31/485; A61K 47/12; A61K 47/10; A61P 25/04

(52) U.S. Cl. CPC A61K 9/7061; A61K 9/0014; A61P 25/04; A61K 47/12; A61K 47/10; A61K 31/485

(57) ABSTRACT

The present invention provides a transdermal patch comprising: a drug-containing layer comprising (R)-dihydroetorphine, or a salt or a hydrate thereof, and a poly(meth)acrylate; and a backing layer.

### FIELD OF THE INVENTION

The present invention relates to a transdermal patch comprising (R)-dihydroetorphine and to a method of making such a transdermal patch. The invention also relates to the use of a transdermal patch in medicine and in particular in a method of providing pain relief or analgesia.

### BACKGROUND

Pain, which can be acute or chronic, is the most common symptom for which patients seek medical advice and treatment. Acute pain is usually self-limited. Chronic pain persists for 3 months or longer and can lead to significant changes in a patient's personality, lifestyle, functional ability and overall quality of life (K. M. Foley, Pain, in Cecil Textbook of Medicine 100-107 (J. C. Bennett and F. Plum eds., 20th ed. 1996)). Pain can also be classified into different acute, subacute and chronic types including nociceptive, inflammatory, neuropathic or mixed pain.

Pain relief occurs in different clinical settings and is critical in the management and treatment of many diseases wherein pain is experienced as a symptom and/or as a side effect. Opioid analgesics form the cornerstone of contemporary treatment of moderate to severe, acute and chronic, pain. The opioid analgesics that are most commonly used to treat pain include morphine, hydromorphone, methadone, levorphanol, fentanyl, oxycodone, and oxymorphone.

In many circumstances it is necessary to provide pain relief for a prolonged or sustained period of time. Sustained pain relief is particularly desirable in patients suffering from moderate to severe chronic pain, e.g. cancer patients. Oral formulations can provide a therapeutic analgesic effect for up to 12, or in a few cases, up to 24 hours but such formulations still require the drug to be readministered at least once or twice a day.

Another approach to sustained delivery of drugs, including analgesics, is transdermal delivery devices such as transdermal patches. Transdermal patches typically comprise a therapeutically active ingredient (e.g. an opioid), an adhesive, optionally a matrix, a backing layer and a release liner. The release liner is removed prior to application of the patch to the skin to expose the adhesive. The adhesive enables the patch to adhere to the skin thereby allowing for passage of the active ingredient from the patch through the skin and into the blood stream.

Transdermal patches have numerous advantages over other routes of administration. These include:
the treatment is comfortable, non-invasive, pain free and convenient
the treatment is well tolerated with high compliance rates
the treatment can potentially be self-administered once patients have been educated on patch use and disposal
the treatment provides a more constant blood concentration of active ingredient than other routes which avoids frequent dosing
the treatment is ongoing regardless of the time of day
the treatment enables a high level of control over the blood concentration of the drug
the drug bypasses the gastrointestinal tract and the liver where it can be destroyed and instead is delivered to the blood stream
the effects of the drug can be terminated by removal of the patch

Many patent applications and literature articles describe patches comprising opioids and in particular buprenorphine and fentanyl. For example, US2007/0298091 describes patches comprising buprenorphine and WO2009/052204 and US2006/0039960 and WO2005/105009 each disclose patches comprising fentanyl.

Two transdermal patches comprising an opioid are commercially available. The BuTrans® or Norspan® patch, for example, comprises 5 mg, 10 mg, or 20 mg of buprenorphine (a partial opioid agonist) and delivers 5 μg/h, 10 μg/h or 20 μg/h over a period of 7 days. It is indicated for the treatment of non-malignant pain of moderate intensity when an opioid is necessary for obtaining adequate analgesia. The Durogesic® Dtrans® patch comprises 2.1, 4.2, 8.4, 12.6 and 16.8 mg of fentanyl and is indicated for the management of chronic pain including chronic pain due to cancer.

The development of commercially viable transdermal patches that provide controlled and sustained release of a drug is not straightforward. To achieve the benefits of transdermal delivery, a transdermal patch that is stable and is able to achieve a sufficient flux of drug through the skin is necessary. It is critical that the drug, and the other constituents, of the transdermal patch does not undergo degradation or change during storage or use. For example, it is important that the drug remain dissolved within the patch throughout its lifetime in order to be deliverable through the skin. Otherwise the flux of drug through the skin will be inconsistent.

The stability of a drug in a transdermal patch is highly dependent on the nature of the drug and the nature of the patch. For instance, the structure of the drug, and its chemical and physical properties, has a significant influence on stability, flux and its interaction with any polymers it is formulated with. It is not possible to substitute one opioid for another opioid in a patch and obtain a commensurate performance. Each drug requires the development of a suitable transdermal patch.

It is also important that the flux of drug through the skin and into the blood stream can be maintained for a prolonged period of time and ideally at least 3 days for a number of the above-described advantages (e.g. high compliance, infrequent dosing, ongoing treatment) of transdermal delivery to be fully realised. To achieve this it is common to include additional ingredients such as permeation enhancers and permeation sustaining agents into transdermal patches to improve control over the permeation of drug. The inclusion of additional ingredients into transdermal patches, however, makes provision of a stable patch yet more complex since the constituents are prone to interacting with the drug. To overcome this problem it is common to provide the drug in specific drug-reservoir layers which are separated from other ingredients to minimise the contact of the drug with them.

Other critical properties of commercially viable patches include: adhesiveness to the skin, stress stability, uniformity of weight and content, flatness and folding endurance.

A wide range of opioid analgesics are known. Opioid agonists include, for example, allylprodine, alphaprodine, anileridine, benzylmorphine, bezitramide, buprenorphine, butorphanol, clonitazene, codeine, desomorphine, dextromoramide, dezocine, diampromide, diamorphone, dihydrocodeine, dihydromorphine, dimenoxadol, dimepheptanol, dimethylthiambutene, dioxaphetyl butyrate, dipipanone, eptazocine, ethoheptazine, ethylmethylthiambutene, ethylmorphine, etonitazene, fentanyl, hydrocodone, hydromorphone, hydromorphodone, hydroxypethidine, isomethadone, ketobemidone, levorphanol, levophenacylmorphan, lofentanil, meperidine, meptazinol, metazocine, methadone, metopon, morphine, myrophine, narceine, nicomorphine, norlevorphanol, normethadone, nalorphine, nalbuphene, normorphine, norpipanone, opium, oxycodone, oxymorphone, pantopon, papavereturn, paregoric, pentazocine, phenadoxone, phendimetrazine, phendimetrazone, phenomorphan, phenazocine, phenoperidine, piminodine, piritramide, propheptazine, promedol, properidine, propoxyphene, propylhexedrine, sufentanil, tilidine, tramadol and pharmaceutically acceptable salts thereof. To date, only buprenorphine and fentanyl have been formulated into commercially available transdermal patches.

Another known opioid analgesic is (R)-dihydroetorphine (R-DHE) (CAS No. 14357-76-7). Its chemical name is 7,8-dihydro-7a[1-(R)-hydroxy-1-methylbutyl]-6,14-endo-ethanotetrahydro-oripavine. Its stereochemical configuration is with 5R, 6R, 7R, 9R, 13S, 14S, 19R and it is shown below.

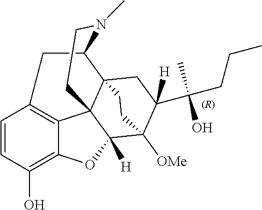

The properties of (R)-dihydroetorphine have been investigated to a far lesser extent than the properties of other opioid analgesics. Clinically it has only been used in humans in China in injectable, and more recently, sublingual form.

There are also relatively few literature reports on the use of (R)-dihydroetorphine. US2005/002997 discloses a transdermal dosage form comprising both a drug and an antagonist to minimise abuse of the dosage form. A long list of possible drugs is disclosed including dihydroetorphine, but, as in the prior art documents mentioned above, the focus of US2005/002997 is on fentanyl. The transdermal dosage form disclosed in US2005/002997 specifically requires the drug to be separated from the adverse agent. Thus typically there exists a drug-containing layer and an adverse agent layer, separated by a barrier which prevents diffusion of the drug and the adverse agent in the absence of solvent. Thus in normal transdermal use, only the drug is transdermally delivered. The drug-containing layer is also required to comprise at least one channel which connects the skin contacting surface with the barrier. The channel enables solvent (e.g. saliva or solvent) to access the adverse agent layer in the event an abuser attempts to extract drug from the transdermal patch.

Two literature articles disclose basic dihydroetorphine containing patches. Chen et al. in Acta Pharmaceutica Sinica 1996 31 (10), 770-774 disclose a patch comprising a dihydroetorphine layer as well as a separate adhesive layer. The adhesive layer primarily comprises polyvinyl alcohol, polyvinyl pyrrolidone, lactose and azone. Ohmori et al. in J. Pharm. Pharmacol. 2000 52, 1437-1449 diclose a patch comprising dihydroetorphine and a styrene-isoprene-styrene block copolymer.

JP-A 10-231248 to TTS Gijutsu Kenkyusho KKrefers to a prototype transdermal device comprising dihydroetorphine and a styrene-isoprene-styrene block copolymer. More specifically JP-A 10-231248 refers to a tape for percutaneous absorption which comprises dihydroetorphine and styrene-isoprene-styrene block copolymer. The purpose of the preparations in JP-A 10-231248 is said to be to provide a sustained therapeutic effect. This is preferably achieved by including a percutaneous absorption enhancer and a percutaneous absorption-sustaining agent in the preparation. The effect of the percutaneous absorption enhancer is to accelerate percutaneous absorption and the effect of the percutaneous absorption-sustaining agent is to sustain absorption.

In the examples of JP-A 10-231248 some preparations are prepared and the rate of dihydroetorphine release is measured. There is, however, no disclosure of a patch which provides prolonged delivery of dihydroetorphine for a clinically useful period of time, e.g. at least 3 days.

JP-A 10-231248 does not therefore disclose a clinically useful transdermal patch

We have found that when prototype transdermal patches comprising a drug-containing layer of (R)-dihydroetorphine and styrene-isoprene-styrene block copolymer, as illustrated in JP-A 10-231248, were prepared and tested, the (R)-dihydroetorphine was found to be highly unstable. Under forced conditions, designed to replicate long-term storage, it was found that (R)-dihydroetorphine, in the presence of styrene-isoprene-styrene block copolymer, had a strong tendency to crystallise out in the drug-containing layer. This is highly undesirable since it was found that the (R)-dihydroetorphine will not redissolve once crystallised. When in crytallised form, however, the (R)-dihydroetorphine is unavailable for transdermal delivery through the skin. Consequently the permeation and flux of (R)-dihydroetorphine is decreased.

### SUMMARY OF INVENTION

Viewed from a first aspect the present invention provides a transdermal patch comprising:
a drug-containing layer comprising (R)-dihydroetorphine, or a salt or a hydrate thereof, and a poly(meth)acrylate; and
a backing layer.

Viewed from a further aspect the present invention provides a transdermal patch comprising:
a drug-containing layer comprising (R)-dihydroetorphine, or a salt or a hydrate thereof, and a pressure sensitive adhesive; and
a backing layer;
wherein said patch is a 3 to 7 day patch.

Viewed from a further aspect the present invention provides a transdermal patch comprising:
a drug-containing layer comprising (R)-dihydroetorphine, or a salt or a hydrate thereof, and a pressure sensitive adhesive; and
a backing layer;
wherein said patch provides a therapeutically effective amount of (R)-dihydroetorphine, or a salt or a hydrate thereof, for at least 72 hours.

Viewed from a further aspect the present invention provides a transdermal patch comprising:
a drug-containing layer comprising (R)-dihydroetorphine, or a salt or a hydrate thereof, and a pressure sensitive adhesive; and
a backing layer;
wherein wherein no crystallisation of (R)-dihydroetorphine, or a salt or hydrate thereof, in the drug-containing layer occurs during storage at 60° C. in a sealed system for at least 1 week.

Viewed from a further aspect the present invention provides a method of making a patch as hereinbefore described comprising:
(i) depositing a composition (e.g. solution) comprising (R)-dihydroetorphine, or a salt or a hydrate thereof, and a poly(meth)acrylate onto a backing layer;
(ii) evaporating said solvent to form a drug-containing layer; and
(iii) optionally applying a release liner to said drug-containing layer.

Viewed from a further aspect the present invention provides a method of making a patch as hereinbefore described comprising:
(i) depositing a composition (e.g. solution) comprising (R)-dihydroetorphine, or a salt or a hydrate thereof, and a poly(meth)acrylate onto a release liner;
(ii) evaporating said solvent to form a drug-containing layer; and
(iii) applying a backing layer to said drug-containing layer.

Viewed from a further aspect the present invention provides a patch comprising (R)-dihydroetorphine for use as a 7 day patch, and in particular for use in treating pain over a period of 7 days.

Viewed from a further aspect the present invention provides a patch comprising (R)-dihydroetorphine for use as a 1 day patch.

Viewed from a further aspect the present invention provides a patch as hereinbefore described for use in medicine.

Viewed from a further aspect the present invention provides a patch as hereinbefore described for use in the treatment of pain.

Viewed from a further aspect the present invention provides a method for the treatment of pain in a subject in need thereof comprising applying a patch as hereinbefore described to the skin of said subject. In particular, in embodiments where the patch is a 7 day patch, it is applied to the skin of the subject for a period of 7 days; where the patch is a 3 day patch, it is applied to the skin of the subject for a period of 3 days; where the patch is a 1 day patch, it is applied to the skin of the subject for a period of 1 day.

### Definitions

As used herein the term “transdermal patch” refers to an adhesive pad capable of delivering (R)-dihydroetorphine, or a salt, or a hydrate thereof, through the skin or mucosal tissues to the blood stream and adhering to the skin. The term transdermal patch also encompasses transdermal plaster, transdermal tape and transdermal disc.

As used herein the term “layer” refers to a continuous body or film of material. Layers do not have any breaks or interruptions therein. Layers may or may not have a uniform thickness. Layers may or may not be planar.

As used herein the term “laminate” refers to a multilayered structure comprising at least two layers connected or bonded together. Preferred patches of the present invention are laminates.

As used herein the term “backing layer” refers to a layer that is a constituent of a patch, which in use of the patch, is remote to the skin. The backing layer covers the drug-containing layer and thereby protects it from exposure to the environment.

As used herein the term “drug-containing layer” refers to a layer comprising (R)-dihydroetorphine, or a salt, or a hydrate thereof, and optionally other active ingredients. In use the drug-containing layer is in contact with the skin.

As used herein the term “pressure sensitive adhesive” refers to an adhesive that requires only minimal pressure, e.g. manual pressure, to stick to the surface of the skin.

As used herein the term “release liner” refers to a removable layer of the patch that is removed prior to application of the patch to skin. The purpose of the release liner is to prevent the patch from loss of drug prior to its application to the skin.

As used herein the term “poly(meth)acrylate” refers to a polymer comprising acrylate and/or methacrylate monomers. These polymers are also often referred to as acrylic acid ester and methacrylic acid ester polymers.

The terms pain relief and analgesia are used herein interchangeably.

### DESCRIPTION OF INVENTION

The transdermal patch of the present invention comprises a drug-containing layer comprising (R)-dihydroetorphine, or a salt or a hydrate thereof, and a poly(meth)acrylate; and a backing layer. In use, the drug-containing layer is in contact with the skin and the backing layer is remote to the skin.

Preferred transdermal patches of the present invention further comprise a release liner which is removable or detachable. When present, the release liner is present on the opposite side of the drug-containing layer to the backing layer. The release liner is removed or detached prior to use of the transdermal patch to expose a surface of the drug-containing layer for contact with the skin. Preferred transdermal patches of the present invention are self-adhering. Thus when the release liner is removed and the patch is applied to the patient's skin, the patch remains attached thereto without there being a need for any separate attachment mechanism, e.g. straps or ties.

The transdermal patch of the present invention may be a drug in adhesive patch or a matrix patch. Preferably the transdermal patch is a drug in adhesive patch, such as a single layer or multi-layer drug in adhesive patch. Single layer drug in adhesive patches are most preferred. Preferably the drug in adhesive layer is continuous. Particularly preferably the drug in adhesive layer does not comprise any channels.

The transdermal patch of the present invention may comprise 2, 3, 4 or 5 layers. Preferred patches comprise 3 or 5 layers and especially preferably 3 layers.

Preferred transdermal patches of the present invention have the structures A, B, C or D comprising (e.g. consisting of) the following layers, wherein the layers are present in the numerical order specified:

(A) (i) a backing layer;
(ii) a drug-containing layer comprising (R)-dihydroetorphine, or a salt or a hydrate thereof, and a poly(meth)acrylate; and
(iii) optionally a release liner.

(B) (i) a backing layer;
(ii) a first drug-containing layer comprising (R)-dihydroetorphine, or a salt or a hydrate thereof, and a poly(meth)acrylate;
(iii) a separating layer;
(iv) a second drug-containing layer comprising a drug; and
(v) optionally a release liner.

(C) (i) a backing layer;
(ii) an adhesive layer;
(iii) a separating layer;
(iv) a drug-containing layer comprising (R)-dihydroetorphine, or a salt or a hydrate thereof, and a poly(meth)acrylate; and
(v) optionally a release liner.

(D) (i) a backing layer;
(ii) a drug-containing layer comprising (R)-dihydroetorphine, or a salt or a hydrate thereof and a poly(meth)acrylate;
(iii) a separating layer;
(iv) an adhesive layer; and
(v) optionally a release liner.

In transdermal patches having the structure (A), (B) or (D), each of the layers is preferably planar. In transdermal patches having the structure (C), the backing layer, the separating layer, the drug-containing layer and, when present, the release liner are preferably planar. The adhesive layer present in structure (C) is preferably non-planar. Preferably the adhesive layer, together with the release liner, surrounds the separating layer and the drug-containing layer, i.e. the separating layer and the drug-containing layer are encapsulated or encompassed.

Particularly preferred transdermal patches of the present invention are those having the structures (A), (B) or (C), more preferably (A) or (C) and still more preferably (A). Preferred transdermal patches comprise a release liner. Preferred transdermal patches do not comprise an adverse agent layer.

The drug-containing layer of the transdermal patch of the present invention comprises (R)-dihydroetorphine. The (R)-dihydroetorphine may be present in the form of a free base or a pharmaceutically acceptable salt. Whether present as a free base or as a pharmaceutically acceptable salt, the (R)-dihydroetorphine may be present in anhydrous form or in the form of a hydrate.

Preferred salts are those that retain the biological effectiveness and properties of (R)-dihydroetorphine and are formed from suitable non-toxic organic or inorganic acids. Acid addition salts are preferred. Representative examples of salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, salicylic acid, methanesulfonic acid, oxalic acid, succinic acid, citric acid, malic acid, lactic acid, fumaric acid and trifluoro acetic acid. The modification of a compound into a salt is a technique well known to chemists to obtain improved physical and chemical stability, hygroscopicity, flowability and solubility of compounds.

Particularly preferably the drug-containing layer comprises (R)-dihydroetorphine in the form of free base.

The drug-containing layer of the transdermal patch of the present invention may comprise (R)-dihydroetorphine, or a salt, or a hydrate thereof, as the sole active ingredient. Alternatively (R)-dihydroetorphine, or a salt, or a hydrate thereof, may be present in combination with another active ingredient. More preferably, however, (R)-dihydroetorphine, or a salt, or a hydrate thereof, is the sole active ingredient present in the drug-containing layer. Still more preferably (R)-dihydroetorphine, or a salt, or a hydrate thereof, is the sole active ingredient present in the patch. Particularly preferably the patch does not comprise an adverse agent.

The drug-containing layer preferably comprises an adhesive and more preferably a pressure sensitive adhesive. The presence of a pressure sensitive adhesive enables the patch to adhere to the skin of a patient. In preferred patches of the present invention no adhesive layer that is separate to the drug-containing layer is required. Instead the adhesive and drug are preferably both incorporated into the drug-containing layer. This simplifies the design and optimisation of the patch.

In a preferred embodiment of the present invention the drug-containing layer comprises a poly(meth)acrylate. The poly(meth)acrylate may be an adhesive and/or a matrix polymer. Preferably the poly(meth)acrylate is an adhesive.

Preferably the poly(meth)acrylate is a copolymer. Preferred copolymers comprise at least two alkyl (meth)acrylate monomers. For example, the copolymer may comprise at least two alkyl acrylate monomers, at least two alkyl methacrylate monomers or may comprise at least one alkyl acrylate monomer and at least one alkyl methacrylate monomer.

In preferred poly(meth)acrylates present in the drug-containing layer of the present invention the alkyl (meth)acrylate monomers comprise 1 to 12 carbon atoms in the alkyl group. Preferably the alkyl (meth)acrylate monomers are selected from methyl acrylate, ethyl acrylate, propyl acrylate, butyl acrylate, isobutyl acrylate, pentyl acrylate, hexyl acrylate, 2-ethylhexyl acrylate, octyl acrylate, isooctyl acrylate, decyl acrylate, dodecyl acrylate, methyl methacrylate, ethyl methacrylate, propyl methacrylate, butyl methacrylate, isobutyl methacrylate, pentyl methacrylate, hexyl methacrylate, 2-ethylhexyl methacrylate, octyl methacrylate, isooctyl methacrylate, decyl methacrylate, dodecyl methacrylate and isomers thereof.

The poly(meth)acrylate may further comprise other monomers. The poly(meth)acrylate may, for example, comprise one or more vinyl ester monomers, e.g. vinyl acetate. Preferably, however, the poly(meth)acrylate does not comprise vinyl ester monomers.

The poly(meth)acrylate may further comprise one or more functionalised monomers. Preferred functionalised monomers are carboxy and hydroxy funtionalised monomers. Preferred carboxy functionalised monomers comprise 3 to 6 carbon atoms. Representative examples of suitable carboxy functionalised monomers include acrylic acid, methacrylic acid, methacrylic acid, itaconic acid, maleic acid, maleic anhydride, and beta-carboxyethyl acrylate. Representative examples of suitable hydroxy functionalised monomers include hydroxyethyl acrylate, hydroxypropyl acrylate, hydroxyethyl methacrylate and hydroxypropyl methacrylate. Preferably, however, the poly(meth)acrylate does not comprise functionalised, e.g. carboxy or hydroxy, functionalised monomers.

The poly(meth)acrylate may further comprise crosslinkable monomers. Representative examples of suitable monomers include glycidyl methacrylate, allyl glycidyl ether and hexanedioldi(methy)acrylate. Preferably, however, the poly(meth)acrylate does not comprise crosslinkable monomers.

The poly(meth)acrylate may further comprise a nitrogen-containing monomer and preferably a N-substituted acryamide or methacrylamide monomer. Representative examples of suitable monomers include N-vinyl pyrrolidine, N-vinyl caprolactam, N-tertiary octyl acrylamide, dimethyl acrylamide, diacetone acrylamide, N-tertiary butyl acryamide, N-isopropyl acrylamide, N-vinyl acetamide and/or N-vinyl formamide. The poly(meth)acrylate may further comprise an amine-containing monomer, e.g. 2-(diethylamino)ethyl methacrylate. Amine-containing monomers impart functionality to the adhesive. Preferably, however, the poly(meth)acrylate does not comprise nitorgen-containing monomers.

Other comonomers that may be present in the poly(meth)acrylate include styrene and nitriles, e.g. acrylonitrile and cyanoethylacrylate. Such comonomers may be incorporated into the polymer to control its glass transition temperature. Preferably, however, the poly(meth)acrylate does not comprise styrene or nitrile monomers.

Preferred poly(meth)acrylate present in the drug-containing layer comprises 40-100% mol of alkyl acrylate monomers and alkyl methacrylate monomers and 0 to 60% mol of another monomer, more preferably 70-100% mol of alkyl acrylate and alkyl methacrylate monomers and 0 to 30% mol of another monomer and still more preferably 90-100% mol of alkyl acrylate and alkyl methacrylate monomers and 0 to 10% mol of another monomer. Still more preferably the poly(meth)acrylate consists of alkyl acrylate monomers and/or alkyl methacrylate monomers. It has been found that this produces the most stable patches.

Suitable alkyl acrylate and/or alkyl methacrylate copolymers for use in the present invention are commercially available from Henkel under the trade name Duro-Tak. These include, for example: Duro-Tak 87-900A, 87-9301, 87-4098 and 87-9088, acryate polymers which are supplied in an organic solvent (ethyl acetate) and have no hydroxy or carboxyl functional groups; Duro-Tak 87-202A and 387-2510/87-2510, acrylate polymers which are supplied in an organic solvent (ethyl acetate) all having —OH functional groups; Duro-Tak 87-208A, 387-2287/87-2287 and 87-4287 acrylate-vinyl acetate polymers which are supplied in an organic solvent (ethyl acetate) solution all having -OH functional groups; and Duro-Tak 387-2516/87-2516 and 387-2525/87-2525 acrylate-vinyl acetate polymers supplied in an organic solvent solution all having —OH functional groups. Particularly preferred copolymers are listed in the table below.

| Tradename | Monomers | Characteristics |
|---|---|---|
| Duro-Tak 87-9301 | Alkyl acrylates; | No OH or |
| | no other | COOH functional |
| | monomers | groups |
| Duro-Tak 87-2510 | Alkyl acrylates and | OH functional groups |
| | hydroxy-containing | present |
| | monomer | |
| Duro-Tak 87-503A | Acrylic-rubber hybrid | |
| Duro-Tak 87-202A | Alkyl acrylates and | OH functional groups |
| | hydroxy-container | present |
| | monomer | |

When mixed with a poly(meth)acrylate in the drug-containing layer, (R)-dihydroetorphine, or a salt, or a hydrate thereof, shows remarkable physical stability, and significantly improved stability compared to drug-containg layers comprising styrene-isobutylene-styrene and polyisobutylene. Thus when present with poly(meth)acrylate, the (R)-dihydroetorphine, or a salt, or a hydrate thereof, shows no tendency to crystallise, even under extreme forced conditions. This is particularly the case when the poly(meth)acrylate consists of alkyl acrylate monomers and/or alkyl methacrylate monomers.

The drug-containing layer of the present invention optionally comprises a second polymer. Representative examples of other polymers include silicone polymers such as polydimethylsiloxane and polymethylphenylsiloxane and rubber polymers such as polyisobutylene and styrene-isoprene-styrene block copolymer. Preferably, however, poly(meth)acrylate is the sole polymer present in the drug-containing layer. This is advantageous as it yields patches having the longest storage capabilities.

The drug-containing layer of the present invention may further comprise a permeation enhancer. Thus in some embodiments the drug-containing layer further comprises a skin permeation enhancer. The permeation enhancer is preferably a C₁₋₂₀ monohydric or polyhydric alcohol, C₂₋₂₀ fatty acid, esters of C₂₋₂₀ fatty acid acids and C₁₋₂₀ monohydric or polyhydric alcohols, urea, pyrrolidine derivative, cyclic monoterpenes, 1-dodecylazacycloheptane-2-one, cyclodextrin or calcium thioglycolate.

Representative examples of permeation enhancers include methyl alcohol, ethyl alcohol, propyl alcohol, isopropyl alcohol, butyl alcohol, heptyl alcohol, octyl alcohol, capryl alcohol, nonyl alcohol, decyl alcohol, undecyl alcohol, lauryl alcohol, tridecyl alcohol, myristyl alcohol, pentadecyl alcohol, cetyl alcohol, hexadecyl alcohol, heptadecyl alcohol, stearyl alcohol, oleyl alcohol, nonadecyl alcohol, eicosyl alcohol, ethylene glycol, propylene glycol, 1,3 butadiol, glycerin, acetic acid, propionic acid, butyric acid, valeric acid, caproic acid, enanthic acid, caprylic acid, pelagonic acid, capric acid, lauric acid, myristic acid, palmitic acid, stearic acid, benzoic acid, salicylic acid, lactic acid, oxalic acid, malonic acid, succinic acid, glutaric acid, adipic acid, maleic acid, fumaric acid, malic acid, tartaric acid, phthalic acid, myristyl lactate, cetyl lactate, lauryl lactate, isopropyl myristate, isopropyl palmitate, butyl stearate, myristyl myristate, urea, thiourea, 2-pyrrolidone, 1-methyl-2-pyrrolidone, 5-methyl-2-pyrrolidone, 1, 5-dimethyl pyrrolidone, 1-ethyl pyrrolidone, menthol, limonene and α-terpenol. Yet further examples of permeation enhancers include oleic acid, triacetin, levulinic acid, dodecanol and lauryl lactate.

Preferably the permeation enhancer is selected from oleic acid, oleyl alcohol, triacetin, levulinic acid, dodecanol and lauryl lactate. Particularly preferably the permeation enhancer is selected from oleic acid, oleyl alcohol and triacetin. These enhancers have been found to increase the flux of (R)-dihydroetorphine, or a salt, or a hydrate thereof, and also to provide patches that are stable, even under forced conditions. A mixture of more than one permeation enhancer may be used. For example, a mixture of two or more of the following may be used: oleic acid, oleyl alcohol, triacetin, levulinic acid, dodecanol and lauryl lactate. In a particular embiodiment when a mixture is used, two or more enhancers selected form oleic acid, oleyl alcohol and triacetin are used.

In more preferred embodiments the drug-containing layer does not comprise a permeation enhancer.

The drug-containing layer may optionally comprise a permeation-sustaining agent. Preferably the permeation sustaining agent is a C₁₂₋₃₂ hydrocarbon, C₁₂₋₃₂ alcohol, glycol, C₆₋₃₂ fatty acid, C₆₋₃₂ fatty acid ester, vegetable oil, animal oil, rubber, polyurethane, silicone resin, water-soluble polymer compound, cellulose, urea, cyclodextrin, thickening agent, clay, gelling agent, suspending agent and emulsifying agent.

Representative examples of permeation-sustaining agents include liquid paraffin, which is a mixture of various hydrocarbons, branched-chain paraffins, solid paraffin, white Vaseline, lauryl alcohol, tridecyl alcohol, myristyl alcohol, pentadecyl alcohol, cetyl alcohol, hexadecyl alcohol, heptadecyl alcohol, steryl alcohol, oleyl alcohol, nonadecyl alcohol, eicosyl alcohol, seryl alcohol, melissyl alcohol, ethylene glycol, propylene glycol, trimethylene glycol, 1,3-butane diol, polyethylene glycol and mixtures obtained by mixing in a suitable ratio polyethylene glycols of a low degree of polymerisation such as Macrogol 400 (trade name) and polyethylene glycols of a high degree of polymerisation, such as Macrogol 4000 (trade name), caproic acid, enanthic acid, caprylic acid, pelargonic acid, capric acid, undecyl acid, lauric acid, tridecyl acid, myristic acid, pentadecyl acid, palmitic acid, heptadecyl acid, stearic acid, oleic acid, nonadecanoic acid, arachidonic acid, linoleic acid, linolenic acid, behenic acid, lignoceric acid, cerotic acid, heptacosanoic acid, montanoic acid, melissic acid, lacceric acid, elaidic acid, brassidic acid, myristyl palmitate, myristyl stearate, myristyl myristate, seryl lignocerate, lacceryl cerotate, lacceryl laccerate, natural waxes of animal origin (e.g. beeswax, whale wax or ceramic wax), vegetable-derived natural waxes (e.g. carnauba wax, candelilla wax), glyceryl monolaurate, glyceryl monomyristearate, glyceryl monostearate, glyceryl mono-oleate, glyceryl dilaurate, glyceryl dimyristate, glyceryl distearate, glyceryl tristearate, glyceryl trimyristae, glyceryl tristearate, castor oil, olive oil, soya oil, sesame oil, almond oil, safflower oil, cottonseed oils, turpentine, hydrogenated vegetable oils, mink oil, egg yolk oil, squalane, squalene, lanolin derivatives, natural rubber, SBS butyl rubber, polyisobutylene, polyvinyl alcohol ether, polyurethane, polyamide, ethylene-vinyl acetate copolymer, dimethyl polysiloxane, polyisoprene rubber, styrene-isoprene-styrene block copolymer, styrene butadiene rubber, polyisobutylene, butylene rubber, polyacrylic acid or salts thereof, acrylic acid ester-acrylic acid copolymer, poly-vinyl alcohol, polyvinyl pyridine, hydroxypropyl cellulose and cross-linked versions thereof, sodium alginate, Arabia gum, pectin, tragacanth gum, ethyl cellulose, hydroxymethyl cellulose, hydroxyethyl starch, bentonite and Veegum HV.

Preferably, however, the drug-containing layer does not comprise a permeation sustaining agent. Particularly preferably the drug-containing layer does not comprise a permeation sustaining agent as described above. This is an advantage of the patch of the present invention. It minimises compatibility issues between components of the patch and simplifies its design and optimisation.

The drug-containing layer of the present invention may further comprise other conventional excipients, e.g. tackifiers, pH regulators, fillers, softeners, antioxidants, and viscosity modifying agents. Such additional excipients are preferably added in an amount of less than 30% wt, more preferably less than 20% wt and even more preferably less than 10% wt based on the total weight of the drug-containing layer.

If the adhesive present in the drug-containing layer does not exhibit its adhesive property in the temperature range at which the system is to be applied, a tackifier is preferably added. Suitable tackifiers include terpene-based resins or petroleum-based resins such as alicyclic saturated hydrocarbon resins. The softening point of the tackifier is preferably 60-160° C. Preferably, however, the drug-containing layer does not comprise a tackifier.

The pH of the drug-containing layer is preferably in the range of 6-8 and more preferably 7-7.8. When the pH of the drug-containing layer is below 6, percutaneous absorption of (R)-dihydroetorphine, or a salt, or a hydrate thereof, will tend to be reduced. When the pH of the drug-containing layer is higher than 8, the risk of skin irritation will tend to increase. The pH of the drug-containing layer may be measured, for example, by placing a sample of the patch with the removable release liner removed, having an actual area of 3.48 cm², in a 20 ml vial and adding 20 ml of purified water to the vial, agitating the vial for 3 days at 150 rpm and using a pH Meter for measurement of the obtained liquid. If the pH is outside of the above range, it may be modified using a pH regulator. Suitable pH regulators include organic or inorganic acids, an organic or inorganic acid metal salt, a metal hydroxide and a metal oxide. Alkali metals and alkaline earth metals may be used as metals for organic or inorganic acid salts. Some specific examples of pH regulators are sodium lactate, sodium acetate, sodium hydroxide, or a combination of an acetic acid salt and acetic acid. Preferably, however, the drug-containing layer does not comprise a pH regulator.

Examples of suitable fillers that may be included in the drug-containing layer of the present invention include colloidal silicon dioxide, bentonite and lactose. Preferably, however, the drug-containing layer does not comprise a filler.

A softener may be included in the drug-containing layer. Representative examples of suitable softeners include liquid paraffin, liquid polybutene, liquid isoprene, squalane and squalene or polar oils including vegetable oils (for example, hydrogenated castor oil, cottonseed oil, palm oil and coconut oil). Preferably, however, the drug-containing layer does not comprise a softener.

An antioxidant may be present in the drug-containing layer to minimise the degradation of (R)-dihydroetorphine, or a salt, or a hydrate thereof, and/or the adhesive. Conventional antioxidants may be employed, e.g. tocopherols, butylated hydroxyanisole, ascorbyl palmitate and ascorbyl stearate. Preferably, however, the drug-containing layer does not comprise an antioxidant.

Examples of suitable viscosity modifying agents that may be present in the drug-containing layer include cellulose derivatives and natural or synthetic gums, such as guar gum and tragacanth. Preferably, however, the drug-containing layer does not comprise viscosity modifying agents.

In preferred patches of the invention the drug-containing layer is non-aqueous, i.e. contains essentially no water. Preferably the water content of the drug-containing layer does not exceed 10% based on the total weight of the drug-containing layer.

Particularly preferably, the drug-containing layer consists of (R)-dihyroetorphine, poly(meth)acrylate and optionally a permeation enhancer.

Preferably the drug-containing layer comprises 1 to 10% wt, and more preferably 3 to 7.5% wt, and still more preferably 4 to 6% wt, dihydroetorphine or salt or hydrate thereof, based on the dry weight of the constituents of the drug-containing layer. Preferably the drug-containing layer comprises 70 to 99% wt poly(meth)acrylate, more preferably 90 to 97.5% wt, and still more preferably 92.5 to 95.5% wt, based on the dry weight of the constituents of the drug-containing layer. Preferably the drug-containing layer comprises 0 to 15% wt and more preferably 5 to 10% wt of a permeation enhancer, based on the dry weight of the constituents of the drug-containing layer.

The backing layer is preferably impermeable to (R)-dihydroetorphine, or a salt, or a hydrate thereof, and any other active agent present in the patch. Preferably the backing layer is occlusive. The backing layer preferably serves as a protective cover and may also provide a support function. Preferably the backing layer is flexible so that it can accommodate movement of the patient without breaking. The backing layer is preferably applied to one side of the drug-containing layer.

The backing layer may be formed from a range of different materials including film, fabric, foamed sheet, microporous sheet, textile fabrics, foil or a laminate of the afore-going. Preferably, however, the backing layer is a film, e.g. a polymer fillm. Particularly preferred backing layers comprise a polyolefin (e.g. high and low density polyethylene, polypropylene), fluoropolymer (e.g. polytetrafluoroethylene), nylon, cellulose derivatives, ethylene-vinyl acetate, vinyl acetate, polyvinylchloride, polyurethane, polyesters (e.g. polyethylene phthalate, polyethylene terephthalate, polybutylene terephthalate or polyethylene naphthalate), metal foils (e.g. aluminium) and laminates of the afore-going.

Preferred backing layers are laminates. Laminates are generally preferred since it is possible to combine materials having different properties to provide laminates having an attractive balance of properties. Particularly preferred laminates comprise a polyolefin, a polyester and a metal.

Suitable backing layers are commercially available from a range of suppliers, e.g. 3M. Scotchpak 9738 is an example of a preferred backing layer.

Preferred patches of the present invention also comprise a removable release liner. The removable release liner is removed prior to application of the patch to a patient. The removable layer is preferably applied to the opposite side of the drug-containing layer to the backing layer.

The release liner preferably comprises polyolefin (e.g. high and low density polyethylene, polypropylene), fluoropolymer (e.g. polytetrafluoroethylene), nylon, cellulose derivatives, ethylene-vinyl acetate, vinyl acetate, polyvinylchloride, polyurethane, polyesters (e.g. polyethylene phthalate, polyethylene terephthalate, polybutylene terephthalate or polyethylene naphthalate) and laminates of the afore-going. Preferably the release liner comprises silicone, fluropolymer or a mixture thereof.

Some preferred release liners comprise polyesters, particularly polyethylene terephthalate. Other preferred release liners comprise a silicone and/or fluoropolymer (e.g. Teflon) coating, particularly preferably on the side of the release liner contacting the drug containing layer. The coating may, for example, be provided on a release liner as described above. The silicone or fluoropolymer coating enables the release liner to be easily removed without damaging the drug-containing layer to which it is attached.

Suitable release liners are commercially available form a range of suppliers, e.g. Loparex and 3M. Loparex Primeliner FL 2000 and Scotchpak 1022 release liners are examples of preferred release liners.

When a separate adhesive layer is present, it preferably comprises a pressure sensitive adhesive. Preferred pressure sensitive adhesives are selected from styrene-based block copolymers, polyvinyl acetates, poly(iso)butylenes, natural and synthetic rubbers, polyurethanes, polyisoprenes, organopolysiloxanes and poly(meth)acrylates. Still more preferably the pressure sensitive adhesive is selected from styrene-based block copolymers, polyisobutylenes, organopolysiloxanes and poly(meth)acrylates and yet more preferably organopolysiloxanes and poly(meth)acrylates. Poly(meth)acrylates are especially preferred. Preferably the same adhesive is present in this layer as in the drug-containing layer.

Representative examples of styrene-based block copolymers include styrene-isoprene-styrene block copolymer, styrene-butadiene-styrene block copolymer, styrene-ethylene/butylene-block copolymer and styrene-isobutylene-styrene block copolymer. Styrene-isobutylene-styrene block copolymers are particularly preferred. Suitable styrene-based block copolymers are commercially available, e.g. from Henkel. Duro Tak 87-6911 is an example of a suitable styrene-based block copolymer.

Polybutylenes may comprise polybutylene and/or polyisobutylene. Polyisobutylenes are preferred. Suitable polyisobutylene polymers are commercially available, e.g. from Henkel. Duro Tak 87-618A is an example of a suitable polyisobutylene.

Organopolysiloxanes that are suitable for use in the present invention include polydimethylsiloxanes and polydimethyldiphenylsiloxanes. Suitable organopolysiloxanes are commercially available from Dow Corning Corporation under the tradename BIO-PSA. BIO-PSA 7-4302 is particularly preferred.

Preferred poly(meth)acrylates are those described above in relation to the drug-containing layer.

When present the separating layer preferably comprises a polymer which is impermeable to (R)-dihydroetorphine, or a salt, or a hydrate thereof, and any other active ingredient present in the patch. Particularly preferred separating layers comprise a polyolefin (e.g. high and low density polyethylene, polypropylene), fluoropolymer (e.g. polytetrafluoroethylene), nylon, cellulose derivatives, ethylene-vinyl acetate, vinyl acetate, polyvinylchloride, polyurethane, polyesters (e.g. polyethylene phthalate, polyethylene terephthalate, polybutylene terephthalate or polyethylene naphthalate), and laminates of the afore-going.

The thickness of the drug-containing layer is preferably 20-150 microns, more preferably 30 to 120 microns and still more preferably 40-100 microns. A drug-containing layer thickness of less than 20 microns will tend to result in insufficient flux of drug through the skin and a thickness of greater than 150 microns will render the patch too thick to be attractive to wear and use.

The backing layer can be any appropriate thickness which will provide the desired protective and support functions. Desirable materials and thicknesses will be apparent to the skilled man but may be in the range 40 to 70 microns. Similarly the removable release liner can be any appropriate thickness which will provide the necessary protection to the adhesive layer prior to application. Desirable materials and thicknesses will be apparent to the skilled man but may be in the range 80 to 120 microns. The skilled man will readily determine suitable thicknesses for any separating and/or adhesives layers present in the transdermal patch.

The total thickness of the patch is preferably 100 to 350 microns, more preferably 150 to 300 microns and still more preferably 200 to 250 microns.

Preferred transdermal patches of the present invention have a skin contacting surface area of 2 to 64 cm², more preferably 4 to 64 cm² and still more preferably 6.25 to 36 cm². The patch may be formed into any shape, e.g. as a square, rectangle, circle or oval. The patch may also have a non-geometric shape.

In preferred transdermal patches of the present invention the concentration of (R)-dihydroetorphine, or salt or hydrate thereof, is 0.01 to 0.50 mg/cm², more preferably 0.1 to 0.45 mg/cm² and still more preferably 0.2 to 0.4 mg/cm². In further preferred transdermal patches the concentration of (R)-dihydroetorphine, or salt or hydrate thereof, is 0.5 to 12 mg/patch, more preferably 1 to 10 mg/patch and still more preferably 2 to 8 mg/patch.

The transdermal patches of the present invention are preferably 3 to 7 day patches. This means that the patches can deliver a therapeutically effective amount of (R)-dihydroetorphine, or a salt, or a hydrate thereof, for 3-7 days before the patch needs to be removed and a new patch put on. Preferably the patch of the invention is a 7 day patch. Such patches are highly desirable since the patient only needs to renew their patch once per week. Hence preferred patches, e.g. when applied to the skin of a patient, provides a therapeutically effective amount of (R)-dihydroetorphine, or a salt or hydrate thereof, for at least 72 hours and more preferably 72-168 hours.

Preferred patches of the invention have a steady state in vitro flux rate of (R)-dihydroetorphine or salt or hydrate thereof of 0.3 to 0.9 μg/cm²/h more preferably 0.5 to 0.9 μg/cm²/h and still more preferably 0.7 to 0.9 μg/cm²/h during a period 22 to 72 hours when tested in a Franz cell using dermatomised human skin (e.g. as determined in the examples). Particularly preferred patches of the invention are 25 cm² and comprise 6.25 mg (R)-dihydroetorphine, or a salt, or a hydrate thereof/patch and have a steady state in vitro flux rate of (R)-dihydroetorphine or salt or hydrate thereof of 0.3 to 0.9 μg/cm²/h, more preferably 0.5 to 0.9 μg/cm²/h and still more preferably 0.7 to 0.9 μg/cm²/h during a period 22 to 72 hours when tested in a Franz cell using dermatomised human skin (e.g. as determined in the examples).

Preferred patches of the present invention are stable to storage. Preferably the patches of the invention are physically stable. Preferably the patches of the invention are chemically stable.

Lack of physical stability may manifest in the occurrence of crystallisation of (R)-dihydroetorphine or a salt or hydrate thereof in the drug-containing layer which can be observed microscopically. Such crystallisation is undesirable because it is highly unlikely that once formed the crystals will redissolve in the drug containing layer. Moreover when (R)-dihydroetorphine, or a salt, or a hydrate thereof, is in the form of crystals it cannot be delivered through the skin.

Preferred patches of the present invention are stable as indicated by no crystallisation of (R)-dihydroetorphine or a salt or hydrate thereof in the drug-containing layer (e.g. as determined by microscopic observation, preferably as described in the examples) during storage at 25° C. and 60% relative humidity in a sealed system for at least 1 week, more preferably 2 weeks and still more preferably 4 weeks. Under these conditions, the most preferred patches may be stable for up to, e.g. 52 weeks.

Preferred patches of the present invention are stable as indicated by no crystallisation of (R)-dihydroetorphine or a salt or hydrate thereof in the drug-containing layer (e.g. as determined by microscopic observation, preferably as described in the examples) during storage at 40° C. and 75% relative humidity in a sealed system for at least 1 week, more preferably 2 weeks and still more preferably 4 weeks. Under these conditions, the most preferred patches may be stable for up to, e.g. 52 weeks.

Further preferred patches of the present invention are stable as indicated by no crystallisation of (R)-dihydroetorphine or a salt or hydrate thereof in the drug-containing layer (e.g. as determined by microscopic observation, preferably as described in the examples) during storage at 6-8° C. in a sealed system for at least 1 week, more preferably 2 weeks and still more preferably 4 weeks. Under these conditions, the most preferred patches may be stable for up to, e.g. 52 weeks.

Preferred patches of the present invention are stable as indicated by no crystallisation of (R)-dihydroetorphine or a salt or hydrate thereof in the drug-containing layer (e.g. as determined by microscopic observation, preferably as described in the examples) during storage at 60° C. in a sealed system for at least 6 days. Under these conditions, the most preferred patches may be stable for up to, e.g. 30 days.

Preferred patches of the present invention adhere to human skin for at least 72 hours, more preferably at least 120 hours and still more preferably at least 168 hours. The patches may, for example, adhere to human skin for 72 to 336 hours, more preferably 96 to 240 hours and still more preferably 120 to 168 hours.

The adhesion of a patch may also be tested by measuring its peel strength from a stainless steel surface using a Zwick/Roell machine as described in the examples. The peel strength of patches of the invention comprising (R)-dihydroetorphine, or a salt, or a hydrate thereof, in their drug containing layer may be compared to identical patches but lacking (R)-dihydroetorphine or salt or hydrate thereof from the drug-containing layer. This enables the relative impact of the (R)-dihydroetorphine, or a salt, or a hydrate thereof, on the adhesiveness of the drug-containing layer to be determined. Preferred patches of the invention have a peel strength of ±30%, more preferably ±25% and still more preferably ±10% of an identical system except for the absence of (R)-dihydroetorphine or salt or hydrate thereof in its drug-containing layer.

In a further embodiment of the present invention the transdermal patch comprises:
a drug-containing layer comprising (R)-dihydroetorphine, or a salt or a hydrate thereof, and a pressure sensitive adhesive; and
a backing layer;
wherein said patch is a 3 to 7 day patch.

In a yet further embodiment of the present invention the transdermal patch comprises:
a drug-containing layer comprising (R)-dihydroetorphine, or a salt or a hydrate thereof, and a pressure sensitive adhesive; and
a backing layer;
wherein said patch (e.g. when applied to the skin of a patient) provides a therapeutically effective amount of (R)-dihydroetorphine, or a salt or hydrate thereof, for at least 72 hours.

In a yet further embodiment of the present invention the transdermal patch comprises:
a drug-containing layer comprising (R)-dihydroetorphine, or a salt or a hydrate thereof, and a pressure sensitive adhesive; and
a backing layer;
wherein wherein no crystallisation of (R)-dihydroetorphine, or a salt or hydrate thereof, in the drug-containing layer (e.g. as determined by microscopic observation, preferably as described in the examples) occurs during storage at 60° C. in a sealed system for at least 1 week.

In these patches the pressure sensitive adhesive is preferably a polymer and more preferably a polymer selected from styrene-based block copolymers, polyvinyl acetates, poly(iso)butylenes, natural and synthetic rubbers, polyurethanes, polyisoprenes, organopolysiloxanes and poly(meth)acrylates. Still more preferably the pressure sensitive adhesive is selected from styrene-based block copolymers, polyisobutylenes, organopolysiloxanes and poly(meth)acrylates and yet more preferably organopolysiloxanes and poly(meth)acrylates. Poly(meth)acrylates are especially preferred.

Representative examples of suitable adhesives are those described above.

The patches of the present invention may be prepared using conventional methods. For instance the patch may be prepared by coating a backing layer with a solution of (R)-dihydroetorphine or a salt or hydrate thereof and pressure sensitive adhesive, e.g. poly(meth)acrylate, in a solvent, removing the solvent from the coated layer to form the drug-containing layer and applying a release liner thereon. In an alternative method the patch is prepared by coating a release liner with a solution of (R)-dihydroetorphine or a salt or hydrate thereof and pressure sensitive adhesive, e.g. poly(meth)acrylate, in a solvent, removing the solvent from the coated layer to form the drug-containing layer and applying a backing layer thereon. Preferred methods further comprise a step of cutting the resulting layered structure into the desired size and/or shape. Preferred solvents for the preparation of the solution of (R)-dihydroetorphine, or a salt, or hydrate thereof, include ethylacetate, hexane, heptane, acetylacetone, toluene, isopropanol, methanol and mixtures thereof. Ethylacetate is a particularly preferred solvent. The preferred drying conditions for removal of the solvent in the coated drug-containing layer are 60 to 120° C. for e.g. 5 to 30 minutes.

The patches of the present invention may be used in medicine and particularly for the treatment of pain. A method for the treatment of pain in patient in need thereof comprises: applying a patch as hereinbefore described to the subject. The patch transdermally delivers a therapeutic amount of (R)-dihydroetorphine, or a salt, or a hydrate thereof, through the skin to the bloodstream. Preferably the patch is applied for at least 72 hours.

### DETAILED DESCRIPTION OF THE INVENTION

Referring to FIG. 1a, it shows a transdermal patch of the present invention that is ready to be placed on the skin of a patient. The patch 1 is a laminate of two layers. A top backing layer 2, which is substantially impermeable to (R)-dihydroetorphine, and a drug layer 3, which comprises (R)-dihydroetorphine or a salt or a hydrate thereof and a poly(meth)acrylate. The backing layer 2 defines the top of the patch and serves as a protective cover for the drug layer 3.

Referring to FIG. 1b, it shows a transdermal patch of the present invention in a form suitable for packaging and storage. The patch 10 is a laminate of three layers. A top backing layer 2, a drug layer 3 comprising (R)-dihydroetorphine or a salt or a hydrate thereof and a poly(meth)acryalte adhesive and a removable release liner 4. Prior to use, the removable release liner 4 is removed to expose the drug layer 3 comprising adhesive. This is applied to the skin of a patient.

FIGS. 2a, 2b and 2c each show alternative patch structures in a form suitable for packaging and storage.

FIG. 2a shows another transdermal patch comprising a single drug layer. Compared to the patch in FIG. 1b, however, the patch comprises an additional adhesive layer 6 and a separating layer 5. The separating layer 5 is formed on top of the drug-containing layer 3 and the adhesive layer 6 is formed around the resulting structure. Thus the adhesive layer 6, together with release liner 4, encompasses or encapsulates the drug-containing layer 3 and the separating layer 5. The backing layer 2 is formed on top of the adhesive layer 6. The release liner 4 contacts the underside of the drug-containing layer 3 and the adhesive layer 6 that surrounds the drug-containing layer. In this arrangement, the drug-containing layer may comprise a reservoir of, for example, a solution of the drug. In this case, there would typically be a membrane 7 through which, in use, the drug passes to reach the skin.

FIG. 2b shows a patch comprising multiple drug layers. Thus the patch comprises a top backing layer 2, a first drug layer comprising (R)-dihydroetorphine or a salt or a hydrate thereof and a poly(meth)acrylate adhesive 6, a separating layer (a rate limiting membrane) 5, a second drug containing layer comprising a drug and a pressure senstitive adhesive 3 and a release liner 4. The drug may optionally be (R)-dihydroetorphine or a salt or a hydrate thereof.

FIG. 2c shows a patch comprising a top backing layer 2, a drug-containing layer comprising (R)-dihydroetorphine or a salt or hydrate thereof and poly(meth)acrylate 3, a separating layer (a rate limiting membrane) 5, an adhesive layer 6 and a release liner 4.

### BRIEF DESCRIPTION OF DRAWINGS

FIGS. 1a and 1b show schematics of transdermal patches of the invention;

FIGS. 2a, 2b and 2c show schematics of alternative transdermal patches of the invention;

FIG. 3 illustrates the method of providing a seed crystal to a transdermal patch;

FIG. 4 shows the amounts of (R)-DHE and buprenorphine permeated across dermatomised human skin in an in vitro permeation model;

FIG. 5 shows the amounts of (R)-DHE and buprenorphine permeated from prototype patches in an in vitro permeation model; and

FIG. 6 shows the amounts of (R)-DHE and buprenorphine permeated from prototype patches in a 7 day in vitro permeation model

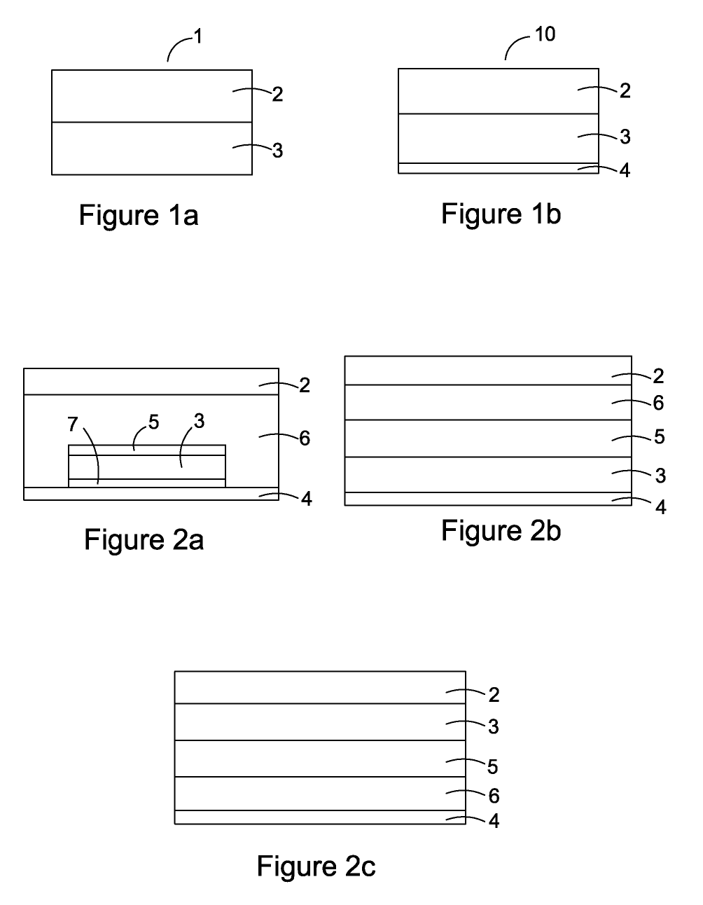

### EXAMPLES

Materials and Equipment

Drug

(R)-DHE was prepared by a synthetic route. Suitable synthetic routes for the preparation of (R)-DHE are known. It is also commercially available.

TABLE 1

| | |
|---|---|
| Name | (R)-Dihydroetorphine (R-DHE) |
| Chemical name | 7,8-dihydro-7a-[1-(R)-hy- |
| | droxy-1-methylbutyl]-6,14-endo- |
| | ethanotetrahydro-oripavine |
| CAS No. | 14357-76-7 |
| Molecular weight | 413.55 |
| Chirality/ | It is a single isomer with 5R, 6R, |
| Stereochemistry | 7R, 9R, 13S, 14S, 19R configuration. |
| Description | White to off white crystalline solid |
| Solubility | Insoluble in water, partially soluble |
| | in ethanol and acetone and readily soluble |
| | in dichloromethane |
| pKₐ1 | 8.2 (tertiary amine) |
| pKₐ2 | 9.5 (aromatic hydroxy) |
| Melting point | 205-207° C. (209° C. by DSC) |
| LogP | 3.5 (neutral species) |

Buprenorphine base, used in comparative testing, was supplied by McFarlan Smith.

Patch Materials

TABLE 2

| Polymer | | | |
|---|---|---|---|
| (solvent system) | Type | Function | Supplier |
| DURO-TAK | Poly(meth)acrylate; | Matrix, | Henkel |
| 87-9301 | no OH or COOH | Adhesive | |
| (Ethylacetate) | functional groups | | |
| DURO-TAK | Poly(meth)acrylate; | Matrix, | Henkel |
| 87-2510 | with OH functional | Adhesive | |
| (Ethylacetate/ | group containing | | |
| hexane) | monomer | | |
| DURO-TAK 87-618A | Polyisobutylene | Matrix, | Henkel |
| (Hexane) | | Adhesive | |
| Bio-PSA 4302 | Silicone | Matrix, | Dow |
| (Ethylacetate) | | Adhesive | Corning |
| DURO-TAK 87-6911 | Styrene-isobu- | Matrix, | Henkel |
| (Toluole/Heptane) | tylene-styrene | Adhesive | |
| | rubber | | |
| DURO-TAK 87-503A | Acrylic-rubber hybrid | Matrix, | Henkel |
| (Ethylacetate/Heptane/ | | Adhesive | |
| Hexane/Pentandione) | | | |
| DURO-TAK 87-202A | Poly(meth)acrylate; | Matrix, | Henkel |
| (Ethylacetate/ | with OH or functional | Adhesive | |
| Isopropanol/ | group containing | | |
| Methanol/Pentandiol) | monomer | | |
| Scotchpak 9738 | Polyethylene/ | Backing layer | 3M |
| | aluminium/ | Occlusive | |
| | Polyester | | |
| Loparex Prime | Silicone coated PET | Release liner | Loparex |
| Liner FL | | | |
| 2000 | | | |
| Scotchpak 1022 | Fluoropolymer coated | Release liner | 3M |
| | poly(ethylene | | |
| | terephthalate) | | |

Solvents

All solvents were obtained from Merck.

Equipment

TABLE 3

| Equipment | Function | Supplier |
|---|---|---|
| Draw down machine with | Solvent casting | Erichsen |
| variable casting knife | | |
| Magnetic stirrer | Mixing | IKA |
| Oven | Drying | Heraeus |
| Marbach Knife | Single patches cutting tool | Marbach |
| Optical Microscope | Identification of (R)-DHE | |
| | crystals | |

Test Methods

Stability Under Different Forced Conditions Test

Supersaturation of a drug can occur in the presence of polymers due to the stabilizing effect of the polymer. During shelf life of the formulation under the destabilizing influence of factors like temperature and humidity the drug can recrystallize. A strong recrystallization may be accompanied by an obvious change in the appearance of the matrix (white spots), a diminished ability to stick to the designated surface or a reduced bioavailability of the drug. On the other hand the formation of crystals can be more subtle and characterized with a microscope in terms of amount, size and shape of the crystals.

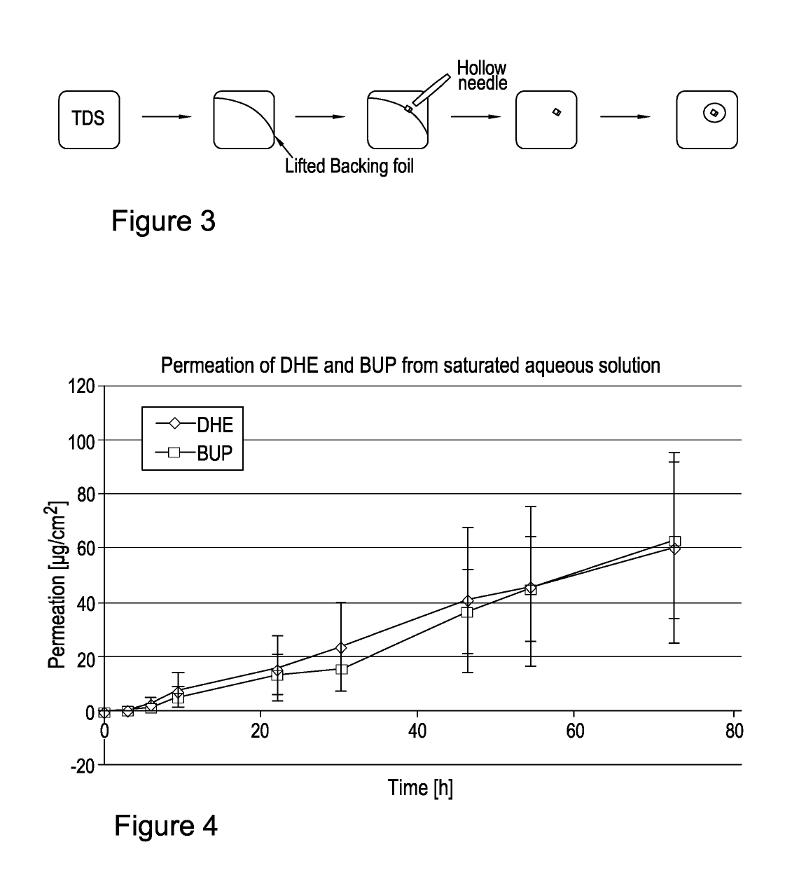

For the standard procedure for the examination of recrystallization six films are punched from the laminate of a tested batch (for example 5 cm²). In order to examine the film without having to peel off the backing layer it is advantageous to use a transparent backing foil. Three of these films are sealed in pouches without any modification. The other three are provided with some seed crystals of the drug as illustrated in FIG. 3. After each examination the films are resealed.

For the rapid test procedure the films are prepared in the same manner but stored without a primary packing material in a petri dish.

Standard Procedure for the Examination of Recrystallization

The films are stored at 25° C./60% relative humidity, 40° C./75% relative humidity or 4-8° C. for up to 4 weeks. After each week all films of one batch are examined after 10 minutes of incubation time at room temperature according to the criteria for the examination of recrystallization.

Criteria for the Examination of Recrystallization
Spots on the surface of the film (Naked Eye)
Size of crystals (Microscope)
Form & Amount of crystals (Microscope)
Stress stability test

The short term stability for six days at 60° C. is a good tool to get a first impression of the compatibility of drug and polymer or other excipients. Therefore the prototypes and corresponding placebo patches are stored in sealed pouches at room temperature and at 60° C. for six days. Placebo samples are important to distinguish correctly between unknowns and placebo signals originating from the matrix. Finally unstressed and stressed placebo and verum samples are analyzed by a content and purity HPLC-UV method to evaluate the drug stability.

HPLC Method for Determination of (R)-DHE

Prepared sample and standard solutions were injected onto a reverse phase HPLC system. Quantification of the active component was against an external referenced standard.

Human Skin Permeation Testing for 3 Days

Skin Preparation

The human skin used for the permeation experiment came from an aesthetic operation. Skin from female donors (breast or abdomen) was supplied from plastic surgery. After arrival, skin was visually checked whether it was without any scars and stretch marks. Female skin has less follicles and hair than male skin.

The layer of 200-500 μm (split-thickness according OECD GUIDANCE NOTES ON DERMAL ABSORPTION) was cut with a dermatome. Round pieces of 2.54cm² were punched out of the skin (permeation area 0.82 cm²).

The diffusion cell consists of a donor chamber and a receptor compartment. The skin is fixed between the compartments. The permeation area (0.82 cm²) of all diffusion cells is equal. The static cell is made of glass. The sampling and volume replacement were manual executed.

In-Vitro Permeation Method 74 h

The patch-samples were placed on the dermatomized skin, the donor compartment of the horizontal Franz-type diffusion cell (5 mL) was filled with acceptor medium (phosphate buffer pH 5.0, 0.1% NaN₃). The permeation took place in a water bath or incubator temperature controlled at 32° C.±1° C. over a time period of 74 h. At each sampling point (3, 6, 8, 22, 30, 46, 54, 74 h) 0.5 mL of the acceptor medium was withdrawn manually and placed into a 0.5 mL vial, 0.5 mL fresh acceptor medium are replaced. The permeation method is listed in the Table below. The content of the drug and of the reference is determined by HPLC-UV. Samples are stored at 2-8° C. until analysis.

| Application of Permeation | Description | |
|---|---|---|
| Source of skin: | Female human skin | |
| Donor: | API patch | |
| No. of samples: | Minimum n = 6 of | |
| | each prototype (if possible) | |
| Acceptor medium: | Phosphate buffer pH 5.0 + 0.1% NaN₃ | |
| Volume: | 5 | mL |
| Permeation area: | 0.82 | cm² |
| Permeation cell: | Horizontal Franz-type diffusion cell | |
| Sampling volume: | 0.5 | mL |
| Volume replacement: | 0.5 | mL |
| Temperature: | 32° C. ± 2° C. | |
| Stirring velocity: | 350 | rpm |

| | |
|---|---|
| Sampling times: | 3, 6, 8, 22, 30, 46, 54 and 74 h |

In-Vitro Permeation Method 168 h

All skin permeations are performed on Franz-cells with a vertical orientation of the dermatomized human skin sample. The investigated patch samples have a size of 0.82 cm².

The diffusion cell consists of a donor chamber and a receptor compartment. The skin is fixed between the compartments. The permeation area (0.82 cm²) of all diffusion cells is equal. The static cell is made of glass. A fully automated sampling device is used to draw samples from the acceptor medium over seven days after 12, 24, 48, 72, 96, 120, 144 and 168 hours. The sample volume was replaced with fresh medium after each sampling procedure. The sampling and volume replacement were executed via auto sampler, all parameters are summarized in the Table below. The Hanson AutoPlus™/Maximizer is a precision syringe-pump sampling system for dissolution testing. The sampling occurs automatically via single-use needles. The sample bottling was done automatically in HPLC-Vials. Determination of the drug content in the samples was done by HPLC with UV detection.

| Application of Permeation | Description | |
|---|---|---|
| Source of skin: | female human skin | |
| Donor: | API patch | |
| No. of samples: | Minimum n = 6 of | |
| | each prototype (if possible) | |
| Acceptor medium: | Phosphate buffer pH 5.0 + 0.1% NaN₃ | |
| Volume: | 5 | mL |
| Permeation area: | 0.82 | cm² |
| Permeation cell: | Vertical Franz-type diffusion cell | |
| Sample Rinse: | 3.0 | mL |
| Collect Only: | 1.5 | mL |
| Replace Media: | 4.5 | mL |
| Temperature: | 32° C. ± 2° C. | |
| Stirring velocity: | 350 | rpm |

| | |
|---|---|
| Sampling times: | 12, 24, 48, 72, 96, 120, 144 and 168 hours |

Peel Strength Test

The purpose of the test was to measure how much force (N) is needed to pull off a sample after a known time, in a defined speed from a known surface. The sample was attached to the stainless steel test plate and fixed by moderate finger pressure. The sample was detached in a 90° angle with 300 mm/min for a defined distance. The force to detach the sample was detected by a force sensor. The force was proportionate to the strip width. Measurement was performed in 30 to 60 seconds. The following equipment was used:

Tensile testing machine: Fa. Zwick, Model BT1-FR2.5TN.D14 with their software

Force sensor: 100 N

Gliding channel and clamp: Art. No. ST/ZUB 16, Fa. Mechanism for measurements in 90° angle, gliding channel refers to DIN 1939

Ground stainless steel plate: KA 044

Separation aid: Tesafix with release liner, material number: 04163 (creped surface) or double faced adhesive tape as fixing aid, extra strong adhesive, e.g. Tesa matrial number: 05696, 2.5×5 cm die-cut strips with release liner

Laminated foil: release liner, 2.5×5 cm

Digital stop watch: Display with seconds

Manual die cutter: Model B/36-AL, TYPE FG 400 of Fa. Hans Naef AG

Form of die cutter: Die cutter, 2.5×5 cm for sample and separation aid strips

Product and machine parameters:

Product size: circular patches, 3.48 cm² area

Velocity of analysis: 300 mm/min

Pre-distance: 5 mm

Distance: 35 mm

Final distance not to be reported: 5 mm

Testing:

Amount of samples: 6 samples are analysed

Conditioning: Samples are equilibriated for 2 h at 23° C.±3° C.

Set up: Gliding channel is installed for measurement in 90° angle

Attachment of sample: 5 mm from the side of the sample was detached from its release liner. 5 mm of the short side of the separation aid was fixed to the exposed drug-containing layer of the sample. The separation aid was folded in the middle of the long length that the adhesive sites were sticking together. The test plate was cleaned with organic solvent before attaching the sample. The release liner was removed from the sample and the sample was fixed without air blowing and folds by finger pressure in the middle of the plate. The folded section of the attached separation aid has a 90° angle to the plate and is fixed to the upper clamp.

Measurement: A stop watch was directly started after adhesive bonding of the sample strip to the test plate. Measurement was begun after a minimum of 30 s and a maximum of 60 s. The force to separate the sample from the plate was measured. Afterwards the test plate was checked for remaining adhesive and, if required, cleaned with organic solvents.

Evaluation: Single values, mean value and standard deviation were calculated

Comparative Patch

A Norspan® transdermal patch (5 micrograms per hour) commercially available from Grunenthal was used in comparative studies. The Norspan® patch contains buprenorphine.

Manufacture of Transdermal Patch Comprising (R)-DHE (0.45% and 4.5% (R)-DHE)

Patches of 3.48 cm² size with a load of 0.025 mg DHE/cm² (total drug load 0.087 mg/patch) were prepared.

(R)-DHE was weighted to a calculated 0.45% drug load (R)-DHE in the dried patch matrix and dissolved in ethylacetate. The matrix solvent system was added and stirred for 30 minutes on a magnetic stirrer to yield a homogenous mixture. The materials used for the preparation of each patch are shown in the tables below. Patches 1-4 comprise poly(meth)acrylate. Patches 5-7 comprise other pressure sensitive adhesives.

TABLE 4a

| | Solid content | Total dry per | Total dry per |
|---|---|---|---|
| Patch 1 | (%) | patch (mg/patch) | patch (%) |
| (R)-DHE | 100.00 | 0.087 | 0.45 |
| DURO-TAK 87-9301* | 38.44 | 19.15 | 99.55 |

*Solvent system as in table 2 above

TABLE 4b

| | Solid content | Total dry per | Total dry per |
|---|---|---|---|
| Patch 2 | (%) | patch (mg/patch) | patch (%) |
| (R)-DHE | 100.00 | 0.087 | 0.45 |
| DURO-TAK 87-2510* | 42.21 | 19.15 | 99.55 |

*Solvent system as in table 2 above

TABLE 4c

| | Solid content | Total dry per | Total dry per |
|---|---|---|---|
| Patch 3 | (%) | patch (mg/patch) | patch (%) |
| (R)-DHE | 100.00 | 0.087 | 0.45 |
| DURO-TAK 87-202A* | 41.46 | 19.15 | 99.55 |

*Solvent system as in table 2 above

TABLE 4d

| | Solid content | Total dry per | Total dry per |
|---|---|---|---|
| Patch 4 | (%) | patch (mg/patch) | patch (%) |
| (R)-DHE | 100.00 | 0.087 | 0.45 |
| DURO-TAK87-503A* | 45.56 | 19.15 | 99.55 |

*Solvent system as in table 2 above

TABLE 4e

| | Solid content | Total dry per | Total dry per |
|---|---|---|---|
| Patch 5 | (%) | patch (mg/patch) | patch (%) |
| (R)-DHE | 100.00 | 0.087 | 0.45 |
| DURO-TAK 87-618A* | 49.53 | 19.15 | 99.55 |

*Solvent system as in table 2 above

TABLE 4f

| | Solid content | Total dry per | Total dry per |
|---|---|---|---|
| Patch 6 | (%) | patch (mg/patch) | patch (%) |
| (R)-DHE | 100.00 | 0.087 | 0.45 |
| Bio-PSA 4302* | 60.86 | 19.15 | 99.55 |

*Solvent system as in table 2 above

TABLE 4g

| | Solid content | Total dry per | Total dry per |
|---|---|---|---|
| Patch 7 | (%) | patch (mg/patch) | patch (%) |
| (R)-DHE | 100.00 | 0.087 | 0.45 |
| DURO-TAK 87-6911* | 57.21 | 19.15 | 99.55 |

*Solvent system as in table 2 above

After mixing, the drug/polymer mixture was hand cast onto a release liner. Loparex Prime Liner FL 2000 was used for the patches having a drug containing layer comprising poly(meth)acrylate, polyisobutylene and styrene copolymers and Scotchpak 1022 was used for the patches having a drug containing layer comprising silicone polymer. Casting was carried out with a casting knife of variable width to achieve a target dry area weight matrix of 55 g/m². The cast was then dried at room temperature for 10 minutes, transferred to a convection oven and dried at 70° C. for 15 minutes and at 100° C. for 5 minutes. Finally the dried casts were hand-laminated with an occlusive backing, Scotchpak 9738, and patches were cut out of the laminate with 0.82, 3.75 and 5 cm² cutting dies.

25 cm² patches comprising 4.5% (R)-DHE and DURO-TAK 87-9301 were also prepared by the same method. Placebo patches, without any (R)-DHE, were also prepared using an identical method.

Results

Stability Under Different, Forced Conditions

The stability of the patches (3.48 cm², 0.45% drug load) was tested under different conditions as listed below and in the following tables according to the test method set out above. Lack of stability was evidenced by the occurrence of re-crystallisation of (R)-DHE in the patches. The level of re-crystallisation was investigated microscopically using an optical microscope. In table 5 below NC indicates no crystallisation was observed microscopically and C indicated crystallisation was observed microscopically. Conditions tested:

Temperature 25° C., relative humidity (RH) 60% and open

Temperature 40° C. and relative humidity (RH) 75% and open

Temperature 40° C., relative humidity (RH) 75% and sealed

Temperature 4-8° C. and sealed

Temperature 60° C. and sealed

[Open means the patch was not contained in a package. Sealed means the patch was enclosed in an impermeable package]

TABLE 5

| | | | | | | | | | | | | | 60° C. |
|---|---|---|---|---|---|---|---|---|---|---|---|---|---|
| | 25° C./60% RH sealed | | | 40° C./75% RH sealed | | | 40° C./75% RH open | | | 4-8° C. sealed | | | sealed |
| Patch | 1 week | 2 weeks | 4 weeks | 1 week | 2 weeks | 4 weeks | 1 week | 2 weeks | 4 weeks | 1 week | 2 weeks | 4 weeks | 6 days |
| 1 | NC | NC | NC | NC | NC | NC | NC | NC | NC | NC | NC | NC | NC |
| 2 | NC | NC | NC | NC | NC | NC | NC | NC | NC | NC | NC | NC | NC |
| 3 | NC | NC | NC | NC | NC | NC | NC | NC | NC | NC | NC | NC | NC |
| 5 | NC | C | C | C | C | C | C | C | C | NC | C | C | C |
| 6 | NC | NC | NC | NC | NC | NC | NC | C | C | NC | NC | NC | C |
| 7 | C | C | C | C | C | C | C | C | C | C | C | C | C |

(R)-DHE patches comprising polyisobutylene and styrene-isobutylene-styrene in their drug-containg layer showed recrystallisation, i.e. lack of stability under the majority of conditions tested. The patch comprising a styrene-isobutylene-styrene adhesive showed recrystallisation after 1 week in all conditions tested. The patch comprising polyisobutylene recrystallised after 1 week at 40° C./75% RH, 6 days at 60° C., 2 weeks at 25° C./65% RH and 1 week at 4-8° C.

The (R)-DHE patches comprising silicone showed sporadic recrystallisation under the most extreme conditions tested, namely 6 days at 60° C. and at 2 weeks in the open at 40° C./75% RH.

The (R)-DHE patches comprising poly(meth)acrylate copolymer were stable in all conditions tested.

Peel Strength Testing From Stainless Steel Surface

The peel strength of each of the patches (3.48 cm², 0.45% (R)-DHE) from a stainless steel surface was tested according to the test method described above with a Zwick/Roell machine in order to discriminate between the patches regarding their adhesion force and to determine if (R)-DHE impacts on the tack strength of the pressure sensitive adhesives. The results are shown in Table 6 below.

TABLE 6

| | | Peel strength Fₙ₌₃ of | |
|---|---|---|---|
| | Peel strength | corresponding placebo patch | Difference in peel |
| Patch | Fₙ₌₃ [N(±Sd)] | [N(±Sd)] | strength (N) |
| 1 | 5.47 (±0.40) | 4.28 (±0.89) | +1.19 |
| 2 | 7.83 (±1.05) | 6.26 (±0.50) | +1.57 |
| 3 | 7.27 (±0.22) | 6.12 (±0.64) | +1.15 |
| 5 | 5.51 (±0.16) | 6.15 (±0.74) | −0.64 |
| 6 | 6.58 (±0.16) | 6.05 (±0.47) | +0.53 |
| 7* | 15.50 (±0.41) | 6.62 (±1.81) | +8.88 |

*Patches were microscopically checked for re-crystallisation prior to testing and the styrene containing patches showed sporadic re-crystallisation

The peel strength was in the same order of magnitude for all patches tested, except for patch 7, the styrene containing patch, which showed some re-crystallisation at the time of testing. It is thought that this may have caused increased peel strength. In all other cases there was no significant difference between the (R)-DHE containing patches and the placebo patches. This shows that the drug does not have a deleterious impact on the performance of the pressure sensitive adhesives.

Permeability Testing

In Vitro Testing

Prior to testing the patch of the present invention, the intrinsic permeability of (R)-DHE across human skin in an in vitro model was tested. A saturated solution of (R)-DHE in phosphate buffered saline (PBS) at pH 5 (10 mg/ml) was added to the donor compartment (500 μl) of a vertical Franz diffusion cell (J Invest Dermatol, 1975, March, 64(3), 190-5) with 5 ml PBS at pH 5 as acceptor medium. The experiment was performed at skin temperature (32° C.). The human skin used was split to a thickness of approximately 500 μm and the permeation area was 1 cm². In intervals of approximately 0, 3, 6, 9.5, 22, 30, 46, 54 and 72 hours samples of 500 μl were drawn manually and the amount of (R)-DHE in the acceptor medium was analysed with HPLC. After sampling, 500 μl PBS at pH 5 were readded to the system. 9 cells were tested.

Buprenorphine was used as a reference compound. 9 cells with buprenorphine were tested equally.

The amounts of (R)-DHE and buprenorphine that permeated across dermatomised human skin were plotted against time and the linear flux rates between 22 and 72 hours were calculated. The results are shown in Table 7 below and in FIG. 4.

Linear regression of the mean data points revealed a linear increase of the permeated (R)-DHE and buprenorphine over time. A lag time of approximately 3 hours was observed for both compounds. The slope of the regression curve shows an equal permeation rate for both compounds (steady state flux>22 hours, 0.875 mg/(cm²*h) for (R)-DHE and 0.893 mg/(cm²*h) for buprenorphine).

TABLE 7

| | Flux | Cumulative amount after |
|---|---|---|
| Compound | [mg/(cm²*h)] | 72 hours [μg] |
| (R)-DHE | 0.875 | 60.59 |
| Buprenorphine | 0.893 | 63.40 |

The results show that (R)-DHE is able to permeate across dermatomised human skin with a linear correlation over 72 hours. The mean calculated permeation rate of 0.875 μg/(cm²*h) is in the same range as that of buprenorphine.

Testing of Patches in Human Skin Permeation Model

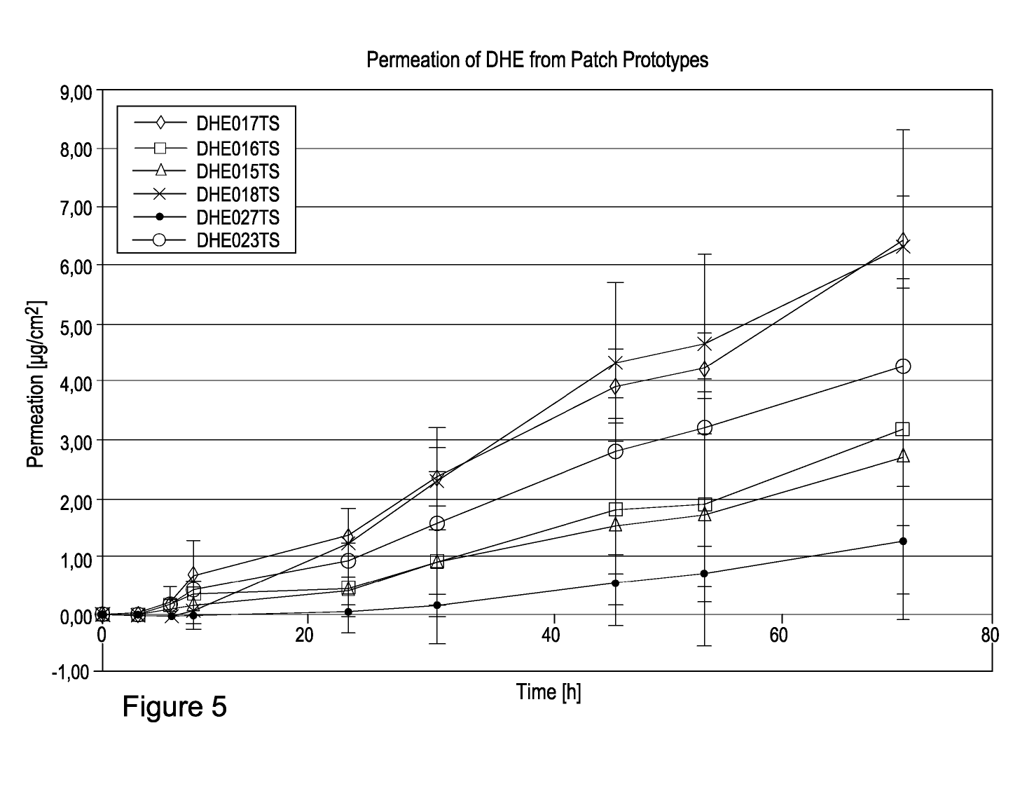

The amounts of (R)-DHE and buprenorphine that permeated from the patches of the invention (3.48 cm², 0.45% API) and the Norspan® patch respectively were plotted against time and the linear flux rates between 22 and 72 hours and cumulative amounts of drug permeated were calculated. The results are shown in Table 8 below and in FIG. 5. Due to the much higher analgesic potency of (R)-DHE compared to buprenorphine, a factor of 1/40 lower steady state permeation rate of (R)-DHE versus buprenorphine was targeted.

TABLE 8

| | Drug | | Flux factor |
|---|---|---|---|
| | permeated/area | Flux > 22 h | Patch of invention |
| Patch | [μg/cm² ± sd] | (μg/(cm²*h)) | vs. Norspan ® |
| 1 | 2.77 (±2.85) | 0.043 | 1/12 |
| 2 | 3.21 (±1.68) | 0.052 | 1/10 |
| 3 | 1.27 (±0.92) | 0.024 | 1/21 |
| 5 | 6.46 (±0.70) | 0.098 | 1/5 |
| 6 | 6.28 (±2.03) | 0.099 | 1/5 |
| 7 | 4.27 (±2.63) | 0.066 | 1/8 |
| Norspan ® | 32.40 (±25.74) | 0.509 | |

Permeation of (R)-DHE across dermatomised human skin into the acceptor medium occurred in all patches tested. The amount of (R)-DHE pemeated was in the single digit μg range for all patches. This was due to the relatively small drug load of the patches (0.087 mg per patch). The permeation factor of (R)-DHE from the patches tested versus Norspan® (5 mg patch) ranged from ⅕ for the polyisobutylene and silicone patches to 1/21 for the poly(meth)acrylate patches. This was at least twice the targeted flux rate based on relative analgesic potency estimates.

Summary

Although the drug load of the patches of the invention tested is low (87 μg per 3.5 cm² patch), (R)-DHE permeation across human skin was measured with HPLC-UV detection from all of the patches tested in a linear manner. Permeation rates were a factor of 1/21 and higher compared to the reference Norpsan® with buprenorphine. This was at least twice the permeation targeted as being an acceptable level.

The patches comprising poly(meth)acrylate were stable in the conditions tested.

Peel strength was comparable for all patches tested, i.e. the presence of (R)-DHE did not influence the tackifying properties of the adhesive polymers tested.

7-Day Permeation Test

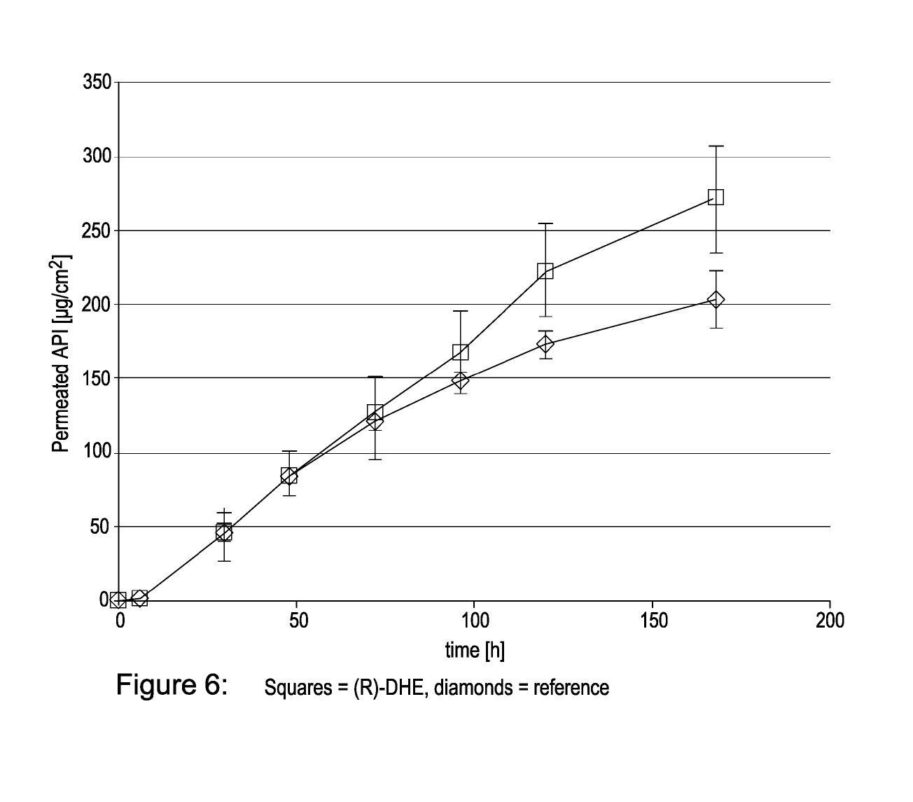

The results from the 7-day permeation test, which was carried out with 25 cm² patches comprising a drug load of 4.5% (R)-DHE are shown in FIG. 6 wherein the red squares represent data for the patch of the invention and the blue diamonds represent the comparative patch. FIG. 6 shows that the patch of the invention delivers (R)-DHE over 168 hours, i.e. 7 days. This follows from the increasing concentration of permeated (R)-DHE.

Manufacture of Transdermal Patches Comprising (R)-dihydroetorphine and Permeation Enhancer

Six different potential enhancers were tested in combination with (R)-dihydroetorphine. Therefore, six basic “drug in polymer” formulations were manufactured for (R)-DHE that each contained one of the six enhancers in a fixed concentration of 5% wt. Patches of these formulations were then tested in a permeation model using human skin as substrate. The patches also underwent a short term stability study to test the compatibility of (R)-DHE, pressure sensitive adhesive and enhancer.

A certain amount of the adhesive, Durotak 87-9301 (in ethylacetate), with a known solids content was weighed in and a calculated amount of enhancer was added. It was assumed that the different enhancers are not volatile and would remain completely in the formulation after the solvents of the adhesive were removed by drying. (The amounts were calculated with regard to the verum formulations, which should contain 90.5% wt adhesive polymer, 5% wt enhancer and 4.5% wt API). The solutions of enhancer in adhesive were divided into two parts, one for the placebo films (needed for content and purity analysis) and one for the verum formulation of (R)-DHE). As (R)-DHE contains water, the purity of (R)-DHE was regarded for the calculation of the drug amount. The calculated amount of (R)-DHE was added to the solution and stirred for several hours to ensure the complete dissolution of the drug. Then the solution was cast on a release liner (siliconized PET) using a casting knife with a defined gap. The solution was dried for 20 min at 70° C. in an oven to remove the solvents from the adhesive. The dry film obtained was then covered with a PET backing film and samples were punched out of this laminate. Area weights were determined and the patches were sealed in pouches. An overview of the different formulations and their compositions is given in Table 9.

TABLE 9

| | Area weight | | API | API content |
|---|---|---|---|---|
| Batch | (mg/cm²) | Enhancer content | content | (mg/cm²) |
| DHE101 | 7.653 | 5.01% oleic acid | 4.50% | 0.3444 |
| DHE102 | 7.388 | 4.96% oleyl alcohol | 4.51% | 0.3330 |
| DHE103 | 7.660 | 5.01% levulinic acid | 4.50% | 0.3451 |
| DHE104 | 7.351 | 5.08% dodecanol | 4.49% | 0.3300 |
| DHE105 | 7.800 | 5.16% lauryl lactate | 4.51% | 0.3514 |
| DHE 106 | 7.599 | 5.03% triacetin | 4.50% | 0.3422 |

Skin Permeation Studies

The prototypes from above were investigated in two sets using the in vitro skin permeation test described above.

In the first set DHE101-DHE104 were tested. Relative flux rates were calculated using Norspan® as reference. The results are summarised in Table 10 below.

It was found that the addition of each of oleic acid and oleyl alcohol resulted in higher flux rates than for formulations that contained dodecanol or levulinic acid. In the case of levulinic acid, the permeation of (R)-DHE was low at the beginning of the experiment and started to increase after 48 hours. The flux rates of oleyl alcohol and dodecanol were almost equal over the first 48 hours. However, after 48 hours the permeation rate decreased for the dodecanol formulation.

TABLE 10

| | Flux | Rank Order |
|---|---|---|
| Sample | (μg/cm²/h) | (highest to lowest flux rates) |
| DHE101 | 0.832 (+/−0.228) | 1 |
| (Oleic acid) | | |
| DHE102 | 0.799 (+/−0.194) | 2 |
| (oleyl alc.) | | |
| DHE103 | 0.673 (+/−0.125) | 3 |
| (levul. acid) | | |
| DHE104 | 0.668 (+/−/0.610) | 4 |
| (dodecanol) | | |
| DHE105 | 0.105 (+/−0.022) | 6 |
| (lauryl lact.) | | |
| DHE102 | 0.241 (+/−0.064) | 5 |
| (triacetin) | | |

In summary the best enhancers for (R)-DHE were oleic acid and oleyl alcohol.

Short-Term Stability Studies

The content and purity of the prototypes was determined by HPLC-UV as described above.

Placebo samples were manufactured (as described above) to distinguish correctly between unknowns and placebo signals originating from the adhesive. A short term stress stability test for six days at 60° C. as described above was used to measure the compatibility of (R)-DHE, DUROTAK 87-9301 and the six enhancers. Therefore, the different prototypes patches and corresponding placebos were stored in sealed pouches at room temperature and at 60° C. The (R)-DHE content and the amount of unknown were quantified by RP-HPLC.

Stability of R-DHE Formulation

The content and purity results of DHE101 to DHE106 are summarized in Tables 12a and 12b. The dimer of (R)-DHE was detected in sample solutions in a concentration of 0.02% to 0.09% and also in the standard solutions. Therefore it is possible that the dimer is not a degradation product but is formed during the analytical process.

The highest amount of impurity was found for unknown RRT 0.95, visible in all formulations with a constant concentration of 0.4%. An exception was DHE101, where RRT 0.95 was not detected. The unknown RRT 0.78 was observed in stressed and unstressed patches in low concentrations of 0.05% to 0.06%.

The levulinic acid containing prototype showed an additional unknown RRT 0.45 after stressing in 0.1%, therefore the sum of impurities increased after the storage at 60° C.

R-DHE is stable over the 60° C-storage and no significant decrease in purity was observed. This is in agreement to the sum of impurities, which is below 0.7% for stressed patches. The levulinic acid containing patch DHE103 had the highest number and total amount of unknowns. The stressed patches with levulinic acid and oleic acid were light yellow.

In summary the six enhancers (5%) tested have no negative influence on (R)-DHE stability.

TABLE 12a

| | | | Content | | | | | | |
|---|---|---|---|---|---|---|---|---|---|
| | | Matrix- | Label | | Unknown | Unknown | Unknown | | Sum of |
| | Verification | weigth | Claim | % A | RRT | RRT | RRT | | Impurities |
| Formulation | Time | [mg] | [%] | [%] | 0.45 | 0.78 | 0.95 | Dimer | [%] ≥0.05% |
| Formulation 1 - 4.5% API | start | 36.92 | 91.40 | 94.58 | n.a. | 0.050 | n.a. | 0.023 | 0.073 |
| DHE101 | | 37.55 | 91.51 | 93.11 | n.a. | 0.054 | n.a. | 0.081 | 0.135 |
| 5% Oleic Acid | | 37.47 | 91.07 | 92.84 | n.a. | 0.058 | n.a. | 0.067 | 0.125 |
| | mean | 37.31 | 91.33 | 93.51 | n.a. | 0.054 | n.a. | 0.057 | 0.111 |
| | s rel [%] | 0.92 | 0.25 | 1.00 | n.a. | 7.407 | n.a. | 53.097 | 29.99 |
| | 6 d 60° C. | 37.39 | 90.15 | 92.11 | n.a. | 0.057 | n.a. | 0.052 | 0.109 |
| | yellow TDS | 39.46 | 94.67 | 91.66 | n.a. | 0.056 | n.a. | 0.041 | 0.097 |
| | | 38.8 | 93.94 | 92.49 | n.a. | 0.063 | n.a. | 0.045 | 0.108 |
| | mean | 38.55 | 92.92 | 92.09 | n.a. | 0.059 | n.a. | 0.046 | 0.105 |
| | s rel [%] | 2.74 | 2.61 | 0.45 | n.a. | 6.453 | n.a. | 12.104 | 6.361 |
| Formulation 2 - 4.5% | start | 36.45 | 97.35 | 98.69 | n.a. | 0.062 | 0.393 | 0.046 | 0.501 |
| DHE102 | | 36.29 | 97.76 | 99.55 | n.a. | 0.051 | 0.405 | 0.052 | 0.508 |
| 5% Oleyl oleate | | 37.00 | 97.66 | 97.53 | n.a. | 0.056 | 0.400 | 0.052 | 0.508 |
| | mean | 36.58 | 97.59 | 98.59 | n.a. | 0.056 | 0.399 | 0.050 | 0.506 |
| | s rel [%] | 1.02 | 0.22 | 1.03 | n.a. | 9.777 | 1.509 | 6.928 | 0.80 |
| | 6 d 60° C. | 37.71 | 101.81 | 99.77 | n.a. | 0.064 | 0.412 | 0.047 | 0.523 |
| | | 35.85 | 97.42 | 100.42 | n.a. | 0.053 | 0.383 | 0.053 | 0.489 |
| | | 37.46 | 100.74 | 99.38 | n.a. | 0.061 | 0.404 | 0.055 | 0.520 |
| | mean | 37.01 | 99.99 | 99.86 | n.a. | 0.059 | 0.400 | 0.052 | 0.511 |
| | s rel [%] | 2.73 | 2.29 | 0.53 | n.a. | 9.584 | 3.748 | 8.058 | 3.69 |
| Formulation 3 - 4.5% | start | 37.82 | 97.03 | 98.01 | n.a. | 0.066 | 0.395 | 0.081 | 0.542 |
| DHE103 | | 38.37 | 99.19 | 98.76 | n.a. | 0.053 | 0.396 | 0.086 | 0.535 |
| 5% Levulinic acid | | 39.28 | 101.25 | 98.48 | n.a. | 0.054 | 0.416 | 0.080 | 0.550 |
| | mean | 38.49 | 99.16 | 98.42 | n.a. | 0.058 | 0.402 | 0.082 | 0.542 |
| | s rel [%] | 1.92 | 2.13 | 0.39 | n.a. | 12.545 | 2.944 | 3.904 | 1.38 |
| | 6 d 60° C. | 38.05 | 98.27 | 98.67 | 0.116 | 0.077 | 0.404 | 0.089 | 0.686 |
| | yellow TDS | 37.71 | 97.54 | 98.82 | 0.111 | 0.068 | 0.404 | 0.093 | 0.676 |
| | | 38.46 | 100.03 | 99.37 | 0.115 | 0.060 | 0.406 | 0.084 | 0.665 |
| | mean | 38.07 | 98.61 | 98.95 | 0.114 | 0.068 | 0.405 | 0.089 | 0.676 |
| | s rel [%] | 0.99 | 1.30 | 0.37 | 2.321 | 12.446 | 0.285 | 5.086 | 1.55 |

TABLE 12b

| | | | Content | | | | | | |
|---|---|---|---|---|---|---|---|---|---|
| | | Matrix- | Label | | Unknown | Unknown | Unknown | | Sum of |
| | Verification | weight | Claim | % A | RRT | RRT | RRT | | Impurities |
| Formulation | Time | [mg] | [%] | [%] | 0.45 | 0.78 | 0.95 | Dimer | [%] ≥0.05% |
| Formulation 4 - 4.5% | start | 36.57 | 64.01 | 63.94 | n.a. | 0.036 | 0.267 | 0.068 | 0.371 |
| DHE104 | | 37.26 | 95.53 | 93.67 | n.a. | 0.055 | 0.455 | 0.109 | 0.619 |
| 5% Dodecanole | | 36.55 | 94.45 | 94.41 | n.a. | 0.067 | 0.453 | 0.085 | 0.605 |
| | mean (n = 2) | 36.79 | 94.99 | 94.04 | n.a. | 0.061 | 0.454 | 0.097 | 0.612 |
| | | 1.10 | | | | | | | |
| | 6 d 60° C. | 35.48 | 92.95 | 95.71 | n.a. | 0.039 | 0.378 | 0.077 | 0.494 |
| | | 37.10 | 96.62 | 95.14 | n.a. | 0.059 | 0.410 | 0.077 | 0.546 |
| | | 37.27 | 97.30 | 95.39 | n.a. | 0.064 | 0.426 | 0.069 | 0.559 |
| | mean | 36.62 | 95.62 | 95.41 | n.a. | 0.054 | 0.405 | 0.074 | 0.533 |
| | s rel [%] | 2.70 | 2.45 | 0.30 | n.a. | 24.498 | 6.040 | 6.214 | 6.45 |
| Formulation 5 - 4.5% | start | 38.88 | 98.74 | 99.15 | n.a. | 0.047 | 0.424 | 0.058 | 0.529 |
| DHE105 | | 38.34 | 94.78 | 96.50 | n.a. | 0.063 | 0.410 | 0.070 | 0.543 |
| 5% Lauryl Lactate | | 38.24 | 95.03 | 97.02 | n.a. | 0.0662 | 0.412 | 0.073 | 0.547 |
| | mean | 38.49 | 96.18 | 97.56 | n.a. | 0.057 | 0.415 | 0.067 | 0.540 |
| | s rel [%] | 0.89 | 2.31 | 1.44 | n.a. | 15.633 | 1.823 | 11.847 | 1.75 |
| | 6 d 60° C. | 36.99 | 93.21 | 98.37 | n.a. | 0.050 | 0.402 | 0.070 | 0.522 |
| | | 37.30 | 93.16 | 97.51 | n.a. | 0.049 | 0.409 | 0.077 | 0.535 |
| | | 39.64 | 98.83 | 97.33 | n.a. | 0.056 | 0.436 | 0.092 | 0.584 |
| | mean | 37.98 | 95.07 | 97.74 | n.a. | 0.052 | 0.416 | 0.0080 | 0.547 |
| | s rel [%] | 3.81 | 3.42 | 0.57 | n.a. | 7.328 | 4.319 | 14.109 | 5.98 |
| Formulation 6 - 4.5% | start | 37.39 | 101.28 | 104.61 | n.a. | 0.056 | 0.419 | 0.063 | 0.538 |
| DHE106 | | 37.52 | 99.37 | 102.28 | n.a. | 0.056 | 0.414 | 0.074 | 0.544 |
| 5% Triacetin | | 38.61 | 102.93 | 102.97 | n.a. | 0.067 | 0.433 | 0.074 | 0.574 |
| | mean | 37.84 | 101.19 | 103.29 | n.a. | 0.060 | 0.422 | 0.070 | 0.552 |
| | s rel [%] | 1.77 | 1.76 | 1.16 | n.a. | 10.644 | 2.334 | 9.030 | 3.49 |
| | 6 d 60° C. | 36.44 | 97.40 | 103.22 | n.a. | 0.060 | 0.417 | 0.062 | 0.479 |
| | | 35.66 | 95.88 | 103.85 | n.a. | 0.063 | 0.412 | 0.056 | 0.468 |
| | | 37.93 | 101.44 | 103.29 | n.a. | 0.070 | 0.433 | 0.051 | 0.484 |
| | mean | 36.68 | 98.24 | 103.46 | n.a. | 0.064 | 0.421 | 0.056 | 0.477 |
| | s rel [%] | 3.14 | 2.92 | 0.33 | n.a. | 7.977 | 2.608 | 9.777 | 0.72 |

Summary

With an enhancer content of 5% it was possible to increase the in vitro permeation rate of (R)-dihydroetorphine by 30-50%. The most promising enhancers were oleic acid and oleyl alcohol. Furthermore, none of the substances tested showed any negative effects on the stability of (R)-DHE in poly(meth)acrylate adhesive. No increase of impurities was observed and the content remained at constant levels after a short term stability study (6 days, 60° C.).

1. A transdermal patch comprising:
a drug-containing layer comprising (R)-dihydroetorphine, or a salt or a hydrate thereof, and a poly(meth)acrylate; and
a backing layer.

2. A patch as claimed in claim 1, further comprising a release liner.

3. A patch as claimed in claim 1, wherein said (R)-dihydroetorphine is in free base form.

4. A patch as claimed in claim 1, wherein said poly(meth)acrylate comprises at least two alkyl (meth)acrylate monomers.

5. A patch as claimed in claim 4, wherein said alkyl (meth)acrylate monomers comprise 1 to 12 carbon atoms in the alkyl group.

6. A patch as claimed in claim 4, wherein said alkyl acrylate monomer is selected from methyl acrylate, ethyl acrylate, propyl acrylate, butyl acrylate, pentyl acrylate, hexyl acrylate, 2-ethylhexyl acrylate, octyl acrylate, isooctyl acrylate, decyl acrylate, dodecyl acrylate, methyl methacrylate, ethyl methacrylate, propyl methacrylate, butyl methacrylate, pentyl methacrylate, hexyl methacrylate, 2-ethylhexyl methacrylate, octyl methacrylate, isooctyl methacrylate, decyl methacrylate, dodecyl methacrylate and isomers thereof.

7. A patch as claimed in claim 1, wherein said poly(meth)acrylate consists of alkyl acrylate monomers and/or alkyl methacrylate monomers.

8. A patch as claimed in claim 1, wherein said drug-containing layer does not comprise a skin permeation enhancer.

9. A patch as claimed in claim 1, wherein said drug-containing layer further comprises a skin permeation enhancer.

10. A patch as claimed in claim 9, wherein said skin permeation enhancer is selected from oleic acid, oleyl alcohol, triacetin, levulinic acid, dodecanol and lauryl lactate.

11. A patch as claimed in claim 10, wherein said skin permeation enhancer is selected from oleic acid and oleyl alcohol.

12. A patch as claimed in claim 1, wherein said drug-containing layer comprises 1 to 10% wt dihydroetorphine or salt or hydrate thereof, based on the dry weight of the constituents of the drug-containing layer.

13. A patch as claimed in claim 1, wherein said drug-containing, layer comprises 70 to 95% wt poly(meth)acrylate, based on the dry weight of the constituents of the drug-containing layer.

14. A patch as claimed in claim 1, wherein said drug-containing layer comprises 0 to 15% wt skin permeation enhancer, based on the dry weight of the constituents of the drug-containing layer.

15. A patch as claimed in claim 1, wherein the concentration of (R)-dihydroetorphine, or salt or hydrate thereof, is 0.01 to 0.5 mg/cm².

16. A patch as claimed in claim 1, wherein the concentration of (R)-dihydroetorphine, or salt or hydrate thereof, is 0.5 to 12 mg/patch.

17. A patch as claimed in claim 1, which is a 3 to 7 day patch.

18. A patch as claimed in claim 1, which provides a therapeutically effective amount of (R)-dihydroetorphine, or a salt or hydrate thereof, for at least 72 hours.

19. A patch as claimed in claim 1 having a mean steady state in vitro flux rate of (R)-dihydroetorphine, or a salt or hydrate thereof, of 0.3 to 0.9 μg/cm²/h during a period 22 to 72 hours when tested in a Franz cell using dermatomised human skin.

20. A patch as claimed in claim 1, wherein no crystallisation of (R)-dihydroetorphine, or a salt or hydrate thereof, in the drug-containing layer occurs during storage at 25° C. and 60% relative humidity in a sealed system for at least 1 week.

21. A patch as claimed in claim 1, wherein no crystallisation of (R)-dihydroetorphine, or a salt or hydrate thereof, in the drug-containing layer occurs during storage at 40° C. and 75% relative humidity in a sealed system for at least 1 week.

22. A patch as claimed in claim 1, wherein no crystallisation of (R)-dihydroetorphine, or a salt or hydrate thereof, in the drug-containing layer occurs during storage at 40° C. and 75% relative humidity in an open system for at least 1 week.

23. A patch as claimed in claim 1, wherein no crystallisation of (R)-dihydroetorphine, or a salt or hydrate thereof, in the drug-containing layer occurs during storage at 6-8° C. in a sealed system for at least 1 week.

24. A patch as claimed in claim 1, wherein no crystallisation of (R)-dihydroetorphine, or a salt or hydrate thereof, in the drug-containing layer occurs during storage at 60° C. in a sealed system for at least 6 days.

25. A transdermal patch of claim 1, further comprising:
a pressure sensitive adhesive;
wherein said patch is a 3 to 7 day patch.

26. A transdermal patch of claim 1, further comprising:
a pressure sensitive adhesive;
wherein said patch is a 1 day patch.

27. A transdermal patch of claim 1 further comprising:
a pressure sensitive adhesive;
wherein said patch provides a therapeutically effective amount of (R)-dihydroetorphine, or a salt or hydrate thereof, for at least 72 hours.

28. A transdermal patch of claim 1, further comprising:
a pressure sensitive adhesive;
wherein wherein no crystallisation of (R)-dihydroetorphine, or a salt or hydrate thereof, in the drug-containing layer occurs during storage at 60° C. in a sealed system for at least 1 week.

29. A method of making a patch as claimed in claim 1, comprising:
(i) depositing a composition comprising (R)-dihydroetorphine, or a salt or a hydrate thereof, and a poly(meth)acrylate onto a backing layer;
(ii) evaporating said solvent to form a drug-containing layer; and
(iii) optionally applying a release liner to said drug-containing layer.

30. A method of making a patch as claimed in claim 1, comprising:
(i) depositing a composition comprising (R)-dihydroetorphine, or a salt or a hydrate thereof, and a poly(meth)acrylate onto a release liner;
(ii) evaporating said solvent to form a drug-containing layer; and
(iii) applying a backing layer to said drug-containing layer.

31-33. (canceled)

34. A method for the treatment of pain in a subject in need thereof comprising applying a patch as claimed in claim 1 to the skin of said subject.

35. A method as claimed in claim 34 wherein the patch is applied to the skin for 7 days.
